(19) United States
(12) Patent Application Publication
(10) Pub. No.: US 2025/0213257 A1
(43) Pub. Date: Jul. 3, 2025

(54) ANGLED GLENOID REAMER

(71) Applicant: Encore Medical, L.P. (d/b/a DJO Surgical), Austin, TX (US)

(72) Inventors: Jeremy McLain, Austin, TX (US); Alysha Robicheaux, Austin, TX (US); Grant Copeland, Austin, TX (US); Morgan Sherry, Austin, TX (US); Trevor Lewis, Austin, TX (US)

(21) Appl. No.: 18/990,569

(22) Filed: Dec. 20, 2024

Related U.S. Application Data
(60) Provisional application No. 63/615,141, filed on Dec. 27, 2023.

(51) Int. Cl. A61B 17/17; A61B 17/16

(52) U.S. Cl. CPC A61B 17/1778; A61B 17/1631

(57) ABSTRACT

In some implementations, apparatuses and methods are provided herein useful to reamer assemblies. In some implementations, a reamer assembly comprises a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, and a boss drill configured to be removably coupled to the drive shaft such that the drive shaft is configured to cause the boss drill to rotate about the first axis of rotation.

### TECHNICAL FIELD

This present disclosure relates generally to orthopedics and, more specifically, the orthopedic equipment.

### BACKGROUND

During surgical procedures, specifically orthopedic surgical procedures, clinicians often remove bone from a patient. For example, a clinician can remove bone by drilling, reaming, cutting, etc. Further, it is often preferred that incisions be as small, and minimally invasive, as possible. While small, minimally invasive incisions are preferred, such incisions provide little room for a clinician to operate. Further, if the incision is small, it can be difficult to insert surgical tools through the incision and have the surgical tools properly aligned. Typically, surgical tools are generally straight. For example, a reaming tool typically includes a straight housing and a reamer secured to the end of the reaming tool. The reamer is driven by a drive shaft and the reamer typically rotates about a same axis as the drive shaft. While this design is easy to manufacture, it has many drawbacks. For example, as noted previously, clinicians typically attempt to use as small of incisions as possible. If the bone to be reamed is angled relative to the incision, the clinician must angle the reaming tool during reaming. This can be difficult with a small incision and with little visibility. Accordingly, a need exists for improved reaming tools.

### BRIEF DESCRIPTION OF THE DRAWINGS

Disclosed herein are implementations of systems, apparatuses, and methods pertaining reamer assemblies. This description includes drawings, wherein:

FIG. 1 is a perspective view of a reamer assembly, according to some implementations of the present disclosure;

FIG. 2A is a first exploded perspective view of the reamer assembly of FIG. 1;

FIG. 2B is a second exploded perspective view of the reamer assembly of FIG. 1;

FIG. 3A is partial side elevation view of the reamer assembly of FIG. 1;

FIG. 3B is partial front elevation view of the reamer assembly of FIG. 1;

FIGS. 4A is a cross-sectional view of a portion of the reamer assembly of FIG. 1 with a guide wire partially inserted;

FIGS. 4B is a cross-sectional view of the portion of the reamer assembly of FIG. 4A with the guide wire fully inserted;

FIG. 5 is a perspective view of the reamer assembly of FIG. 1 in use on a patient, according to some implementations of the present disclosure;

FIG. 6 is a perspective view of a reamer assembly, according to some implementations of the present disclosure;

FIG. 7 is a perspective view of a housing and drive shaft of the reamer assembly of FIG. 6 with a reamer removed, according to some implementations of the present disclosure;

FIG. 8 is an exploded view of the drive shaft and housing of the reamer assembly of FIG. 6, according to some implementations of the present disclosure;

FIG. 9 is a second exploded view of a portion of the reamer assembly of FIG. 6, according to some implementations of the present disclosure;

FIG. 10 is a perspective view of the reamer for use with the reamer assembly of FIG. 6, according to some implementations of the present disclosure;

FIG. 11 is an exploded view of the reamer of FIG. 8, according to some implementations of the present disclosure;

FIG. 12A is a first perspective view of a reamer assembly, according to some implementations of the present disclosure;

FIG. 12B is a second perspective view of the reamer assembly of FIG. 12A; FIG. 13A is a first exploded view of the reamer assembly of FIG. 12A;

FIG. 13B is a second exploded view of the reamer assembly of FIG. 12A;

FIG. 14 is a cross-sectional view along line 14-14 of a first portion of the reamer assembly of FIG. 12A;

FIG. 15 is a cross-sectional view along line 15-15 of a second portion of the reamer assembly of FIG. 12A;

FIG. 16A is a first perspective view of a handle for use with a reamer assembly, according to some implementations of the present disclosure;

FIG. 16B is a second perspective view of the handle of FIG. 16A;

FIG. 17A is a first exploded view of the handle of FIG. 16A;

FIG. 17B is a second exploded view of the handle of FIG. 16A;

FIG. 18A is a disassembled perspective view of the handle of FIG. 16A and the reamer assembly of FIG. 12A in which a movable protrusion of the handle is in an extended position, according to some implementations of the present disclosure;

FIG. 18B is a partial cross-sectional view of the handle and the reamer assembly of FIG. 18A;

FIG. 19A is a perspective view of the handle and the reamer assembly of FIG. 18A in which the movable protrusion is in a retracted position, according to some implementations of the present disclosure;

FIG. 19B is a partial cross-sectional view of the handle and the reamer assembly of FIG. 19A;

FIG. 20A is an assembled perspective view of the handle and the reamer assembly of FIG. 18A in which the movable protrusion is in the retracted position, according to some implementations of the present disclosure;

FIG. 20B is a partial cross-sectional view of the handle and the reamer assembly of FIG. 20A;

FIG. 21A is an assembled perspective view of the handle and the reamer assembly of FIG. 18A in which the movable protrusion is in the extended position, according to some implementations of the present disclosure;

FIG. 21B is a partial cross-sectional view of the handle and the reamer assembly of FIG. 21A;

FIG. 22 is a flow chart depicting example operations for coupling a glenoid implant, according to some implementations.

### DETAILED DESCRIPTION

Generally speaking, pursuant to various implementations, systems, apparatuses, and methods are provided herein useful to reamer assemblies. In some implementations, a reamer assembly comprises a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, and a boss drill configured to be removably coupled to the drive shaft such that the drive shaft is configured to cause the boss drill to rotate about the first axis of rotation.

As previously noted, clinicians typically use as small of incisions as possible when operating on a patient. Additionally, when performing orthopedic surgical procedures, clinicians often need to remove bone at an angle to the incision and/or incision site. Typical reaming tools are generally linear, with the reamer rotating about the same axis as the drive shaft. While such a reaming tool style is easy to manufacture, it is difficult to use in practice. For example, it is often necessary to ream bone at an angle relative to the patient. Due to the small incisions, it can be difficult to properly orient the reaming tool to ream at the appropriate angle.

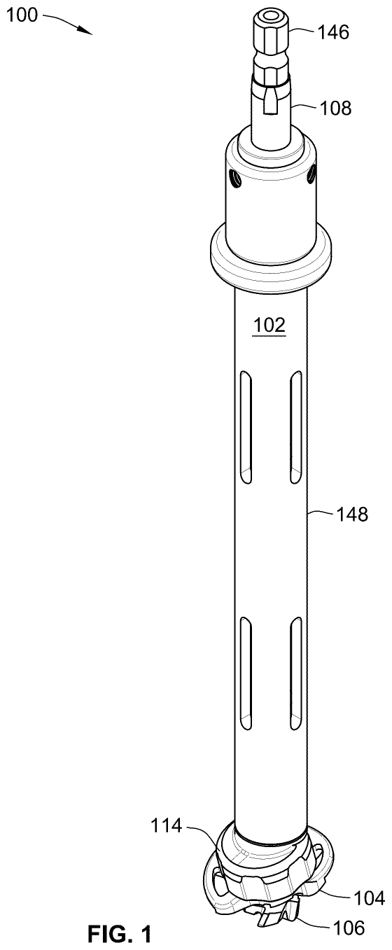

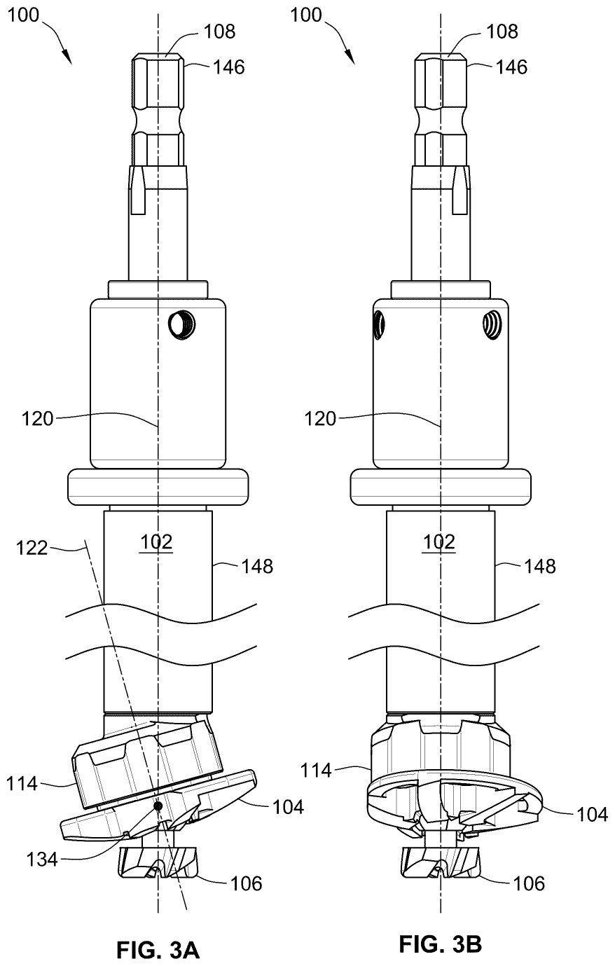

Described herein are reamer assemblies that seek to minimize, if not eliminate, the drawbacks of current reaming tools. In one implementation of the present disclosure, a reamer assembly includes a housing, a drive shaft, and a reamer. The drive shaft is inserted through the housing and rotates about a first axis of rotation. The reamer is driven by the drive shaft. However, the reamer does not rotate about the first axis of rotation (i.e., the axis of rotation of the drive shaft). Instead, the reamer rotates about a second axis of rotation (i.e., the axis of rotation of the reamer) that is different than the first axis of rotation. That is, the second axis of rotation is at a non-zero angle to the first axis of rotation. Additionally, in some implementations, the reamer assembly can include a boss drill. The discussion of FIGS. 1 and 3A-3B provide an overview of such a reamer assembly.

FIG. 1 is a perspective view of a reamer assembly 100, according to some implementations of the present disclosure, and FIGS. 3A and 3B are perspective views of a portion of reamer assembly, according to some implementations. As depicted in FIGS. 1 and 3A-3B, the reamer assembly 100 generally includes a housing 102, a drive shaft 108, a reamer 104, and a boss drill 106. It should be noted that, in some implementations, the reamer assembly 100 may include greater or fewer components than those depicted in FIG. 1. As one example, the reamer assembly 100 depicted in FIG. 1 may not include the boss drill 106 in some implementations, though otherwise be substantially similar.

The housing 102 includes a channel extending therethrough. The channel defines a longitudinal axis 120. When in an assembled state, the drive shaft 108 is at least partially positioned in the channel. The drive shaft 108 is configured to rotate, for example, via a tool (not shown). In one implementation, the drive shaft 108 includes a mating mechanism 146 located at a proximal end of the drive shaft 108. In such implementations, the tool mates with the drive shaft 108 via the mating mechanism 146. As previously noted, the drive shaft 108 is configured to rotate. The drive shaft 108 rotates about a first axis of rotation. In one implementation, the first rotational axis is coincident with, or parallel to, the longitudinal axis 120.

The housing 102 can be “divided” into two sections: an upper housing 148 and a lower housing 114. Though the housing 102 can be “divided” into two section, physical separability of the two sections is not required. For example, the upper housing 148 and the lower housing 114 can be monolithically formed, independently formed and removably coupled (e.g., via fasteners), independently formed and irremovably coupled (e.g., via welds), etc. In one implementation, the lower housing 114 includes an angled face relative to the upper housing 148. That is, the distal end (i.e., the end opposite the mating mechanism 146 of the drive shaft) is not normal to the longitudinal axis 120. Such angled geometry of the lower housing 114 provides an angled axis of rotation of the reamer 104.

The reamer 104 is coupled to the housing 102. Specifically, the reamer 104 is coupled to the lower housing 114. The reamer 104 can be removably or irremovably (i.e., semi-permanently) coupled to the housing 102. As previously noted, the lower housing 114 has an angled geometry at its end. In one implementation, the reamer 104 seats within the lower housing 114. The drive shaft 108 is configured to engage the reamer 104 and cause the reamer 104 to rotate. Because the reamer 104 is angled relative to the drive shaft 108 and thus the first rotational axis, the reamer 104 rotates about a second axis of rotation 122 that is different than the first axis of rotation. That is, the second axis of rotation 122 is neither coincident nor parallel to the first axis of rotation.

As previously noted, in some implementations of the present disclosure, the reamer assembly 100 also includes the boss drill. In such implementations, the boss drill 106 extends below the lower housing 114. Additionally, in some implementations, the boss drill 106 extends beyond the distal surface of the reamer 104. The boss drill 106 is configured to be coupled to the drive shaft 108. The boss drill 106 can be removably or irremovably (i.e., semi-permanently) coupled to the drive shaft 108. The drive shaft 108 is configured to cause the boss drill 106 to rotate. For example, the drive shaft 108 can cause the boss drill 106 to rotate about the longitudinal axis 150 (i.e., about an axis that is coincident with, or parallel to, the first rotational axis). In such implementations, the boss drill 106 rotates about an axis that is different than the axis about which the reamer 104 rotates.

While the discussion of FIGS. 1 and 3A-3B provides an overview of a reamer assembly, the discussion of FIGS. 3A and 3B provide additional detail regarding such a reamer assembly.

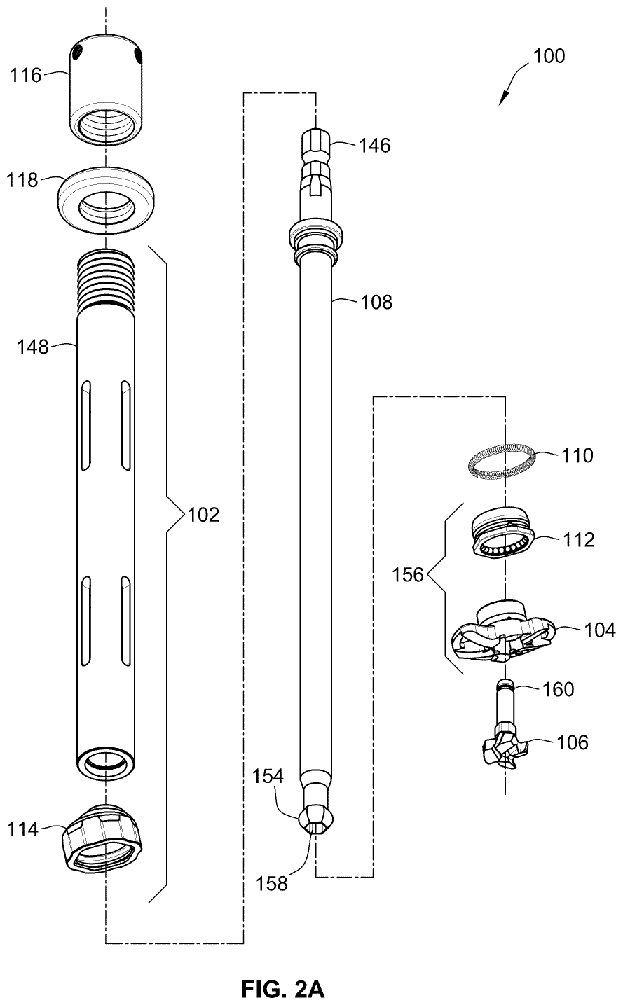

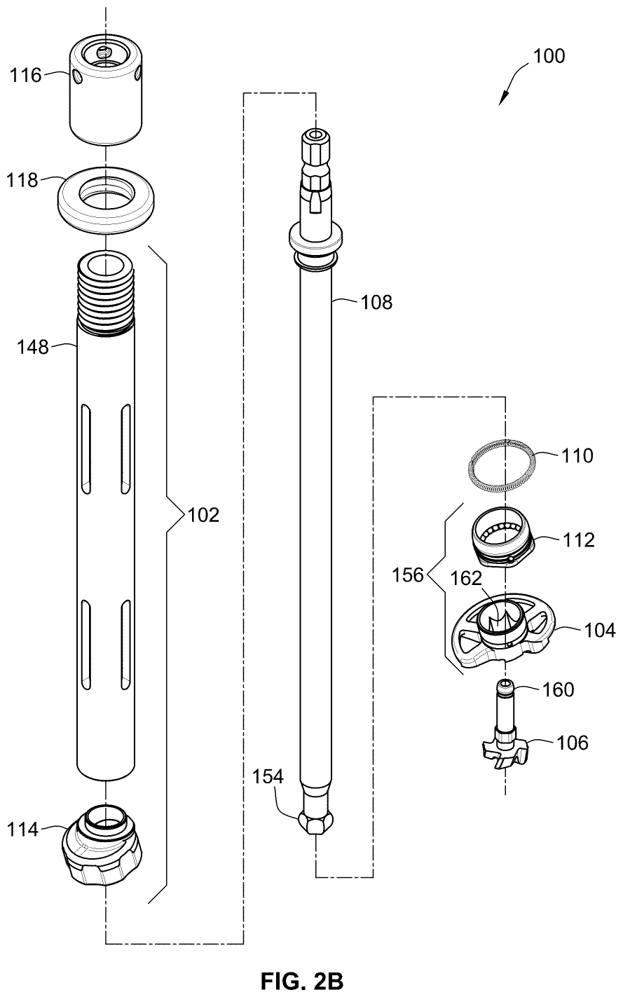

FIGS. 2A and 2B are exploded views of a reamer assembly 100, according to some implementations of the present disclosure. In the example depicted in FIGS. 2A and 2B, the reamer assembly 100 generally includes an upper collar 116, a spacer 118, a housing 102, a drive shaft 108, a first retentive member 110, a bearing assembly 112, a reamer 104, and a boss drill 106. While FIGS. 2A and 2B depict each of the upper collar 116, spacer 118, housing 102, drive shaft 108, first retentive member 110, bearing assembly 112, reamer 104, and boss drill 106, it should be noted that the reamer assembly 100 can include greater, or fewer, components and the components depicted in FIGS. 2A and 2B are but an example for illustrative purposes.

As indicated in FIGS. 2A and 2B, the spacer 118 seats on the upper housing 148 beneath the collar 116 and the lower housing 114 seats on the upper housing 148. The upper housing 148 and the lower housing 114 form the housing 102. The drive shaft 108 is inserted into the housing 102 via a channel 164 within the housing 102. The reamer 104 seats in the bearing assembly 112 to form a reaming head assembly 156. The reaming head assembly 156 couples to the housing 102. Though in the example provided in FIGS. 2A and 2B depicts the bearing assembly 112 as being a component that is distinct from the housing 102, implementations are not so limited. For example, in some implementations, the housing 102 (e.g., the lower housing 114) can include an integral bushing upon which the reamer 104 rotates. As another example, the bearing assembly 112 may be integral to the reamer 104 such that the reamer assembly 156 is a single component. Further, in some implementations of the present disclosure and as depicted in FIGS. 2A and 2B, the reaming head assembly 156 (and/or the bearing assembly 112) can be retained within and/or on the housing 102 via the retentive member 110. As but one example, and as depicted in FIGS. 2A and 2B, the retentive member 110 is a spring (e.g., a coil spring). The retentive member 110 sits in a recess in an inner surface of the housing 102 (e.g., in the lower housing 114) and a recess in an outer surface of the bearing assembly 112 and/or the reamer 104. The boss drill 106, in implementations including the boss drill 106, is coupled to the drive shaft 108.

As previously noted, the drive shaft 108 is configured to rotate about a first axis of rotation. As depicted in FIGS. 2A and 2B, though not required, the proximal end of the drive shaft 108 includes a mating mechanism 146. In implementations in which the drive shaft 108 includes the mating mechanism 146 (and those in which the drive shaft 108 does not include a mating mechanism), a tool can couple to the drive shaft 108 via the mating mechanism 146 to impart rotational motion on the drive shaft 108.

In some implementations of the present disclosure, and as depicted in the example provided in FIGS. 2A and 2B, the distal end of the drive shaft 108 includes a second mating mechanism 154. The second mating mechanism 154 can mate with an internal mating mechanism 162 of the reamer 104. The second mating mechanism 154 and the internal mating mechanism 162 of the reamer 104 can take any suitable form. For example, the second mating mechanism 154 and the internal mating mechanism 162 of the reamer 104 can have corresponding geometries and/or features. As one example, the second mating mechanism 154 can have a hexagonal geometry (e.g., a ball hex) and the internal mating mechanism 162 of the reamer 104 can be an internal hex. In the example provided above, the drive shaft 108 can impart rotational motion on the reamer 104 about an axis that is not coincident or parallel to the rotational axis of the drive shaft (i.e., the first rotational axis). Though the example of a ball hex for the second mating mechanism 154 is provided, any suitable mating mechanism(s) can be employed.

Further, in some implementations of the present disclosure, and as depicted in the example provided in FIGS. 2A and 2B, the distal end of the drive shaft 108 includes an internal mating mechanism 158 of the drive shaft 108. The internal mating mechanism 158 of the drive shaft 108 can mate with an external mating mechanism 160 of the boss drill 106. The internal mating mechanism 158 of the drive shaft 108 and the external mating mechanism 160 of the boss drill 106 can take any suitable form. For example, the internal mating mechanism 158 of the drive shaft 108 and the external mating mechanism 160 of the boss drill 106 can have corresponding geometries and/or features. As one example, the internal mating mechanism 158 of the drive shaft 108 can be an internal hex and the external mating mechanism 160 of the boss drill 106 can be an external hex. As another example, the internal mating mechanism 158 of the drive shaft 108 can include a number of protrusions and the external mating mechanism 160 of the boss drill 106 can include a corresponding number (i.e., the same number as, fewer than, or greater than) of detents that are arranged to accommodate mating of the two components. Regardless of the type of mating mechanism(s) employed, the drive shaft 108 is configured to impart rotational motion on the boss drill 106.

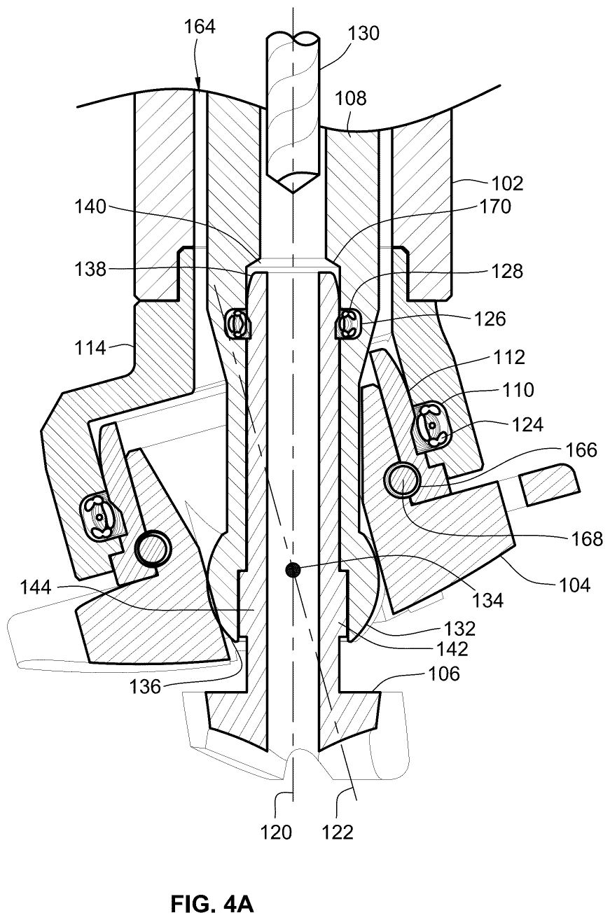

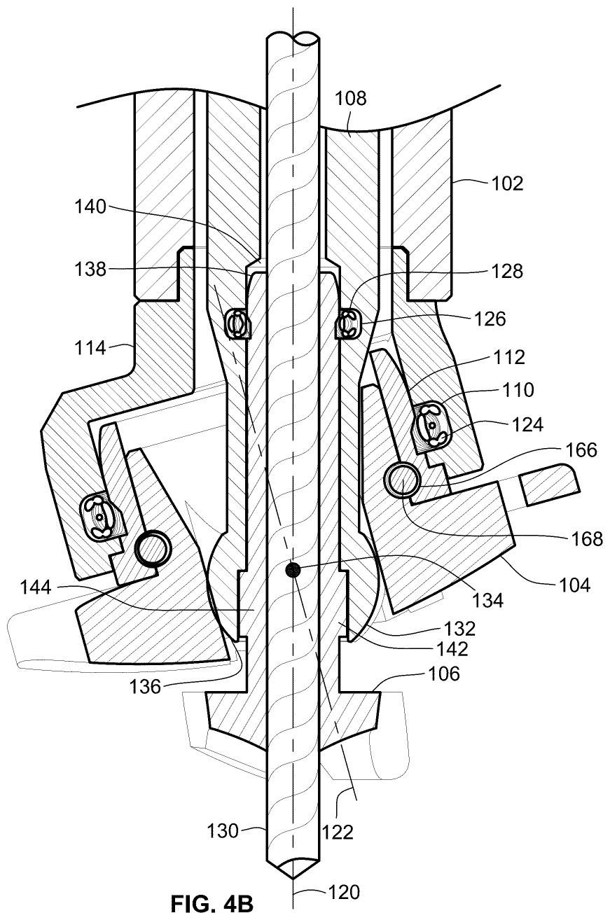

While the discussion of FIGS. 2A and 2B provides additional detail regarding a reamer assembly, the discussion of FIGS. 4A and 4B provides additional detail regarding the coupling of various components of a reamer assembly.

FIGS. 4A and 4B are sectional views of a portion of a reamer assembly 100, according to some implementations of the present disclosure. As depicted in FIGS. 4A, the reamer assembly 100 includes a housing 102, a drive shaft 108, a reamer 104, and a boss drill 106. FIGS. 4A and 4B both depicts a guide wire 130 (e.g., a K-wire, pin, etc.). In FIG. 4A, the guide wire 130 is depicted as being located within the drive shaft 108. For example, the guide wire 130 has been placed and then the reamer assembly has been inserted over the guide wire 130. In FIG. 4B, the guide wire 130 is depicted as extending beyond the boss drill 106 (i.e., after insertion through the drive shaft 108). It should be noted that, in the implementation depicted in FIGS. 4A and 4B, the drive shaft 108 is cannulated, though such is not required in any implementation. Further, the features and components shown in FIGS. 4A and 4B can be utilized in any other implementation, with or without a cannulated drive shaft.

As previously noted, the housing 102 includes a channel 164, through which the drive shaft 108 is positioned. The channel 164 extends through the housing 102 and defines a longitudinal axis 120. The drive shaft 108 rotates about a first rotational axis, the first rotational axis being coincident with the longitudinal axis 120. As previously noted, the lower housing 114 is angled relative to the upper housing 148. Due to this angled geometry, the reamer 104 rotates about a second rotational axis 122 that is different from the first rotational axis (and the longitudinal axis 120). In some implementations, and as indicated in FIGS. 3A, 4A, and 4B, an intersection 134 of the first longitudinal axis (and the first rotational axis) is located within the reamer 104.

As discussed briefly with respect to FIGS. 2A and 2B, mating mechanisms and retentive members can be used to secure components of the reamer assembly 100 to one another. Specifically, as depicted in more detail with respect to FIGS. 4A and 4B, a first retentive member 110 can be used to secure the reamer 104, reamer assembly 156, and/or bearing assembly 112 to the housing 102. As depicted in FIGS. 4A and 4B, the reamer 104 (or reamer assembly 156 or bearing assembly 112) and/or housing 102 includes a first retentive groove 124 (e.g., one of the two components can include a groove, both of the components can include a groove, both of the components can include a portion of a groove, etc.). The first retentive member 110 seats in the first retentive groove 124, aiding in securing the reamer 104, reamer assembly 156, and/or bearing assembly 112 to the housing 102. Similarly, a second retentive member 128 can be used to secure the boss drill 106 to the drive shaft 108. As depicted in FIGS. 4A and 4B, the boss drill 106 and/or the drive shaft 108 can include a second retentive groove 126 (e.g., one of the two components can include a groove, both of the components can include a groove, both of the components can include a portion of a groove, etc.). The second retentive member 128 seats in the second retentive groove 126, aiding in securing the boss drill 106 to the drive shaft 108. Similarly, a third retentive member can be used to secure the reamer 104 to the bearing assembly 112. As depicted in FIGS. 4A and 4B, the reamer 104 and/or bearing assembly 112 include a third retentive groove 166 (e.g., one of the two components can include a groove, both of the components can include a groove, both of the components can include a portion of a groove, etc.). The third retentive member 168 seats in the third retentive groove 166, aiding in securing the reamer 104 to the bearing assembly 112. Each of the retentive members can be of any suitable type. For example, the retentive members can be springs, clips, rings, fasteners, etc. Further the retentive members need not all be of the same type, and a single retentive member can be of multiple types.

In some implementations of the present disclosure, in addition to or in lieu of the second retentive member 128 and the second retentive groove 126, the drive shaft 108 and/or boss drill 106 can include features to aid in securing and/or locating the boss drill 106 to/within the drive shaft 108. For example, in some implementations, the boss drill 106 and/or drive shaft 108 can be dimensioned to limit and/or control the depth of the boss drill 106 in the drive shaft 108. In the example depicted in FIGS. 4A and 4B, an inner diameter of the drive shaft 108 is not uniform. Rather, the inner surface of the drive shaft 108 includes a ledge 140. In the proximal direction from the ledge 140, the inner diameter of the drive shaft 108 is smaller than the inner diameter of the drive shaft 108 in the distal direction from the ledge 140. The boss drill includes a shank 138 and a cutting element 172. The cutting element 172 is located at the distal end of the shank 138. The shank 138 has a diameter such that it can be inserted within the drive shaft 108. However, the diameter of the shank 138 is greater than an inner diameter of the drive shaft 108 proximal of the ledge 140. Accordingly, in the example depicted in FIGS. 4A and 4B, the ledge 140 acts as a physical stop that prevents the boss drill 106 from being inserted too deeply into the drive shaft 108. That is, the shank 138 of the boss drill 106 will physically contact the ledge 140 to limit the depth at which the boss drill 106 can be inserted into the drive shaft.

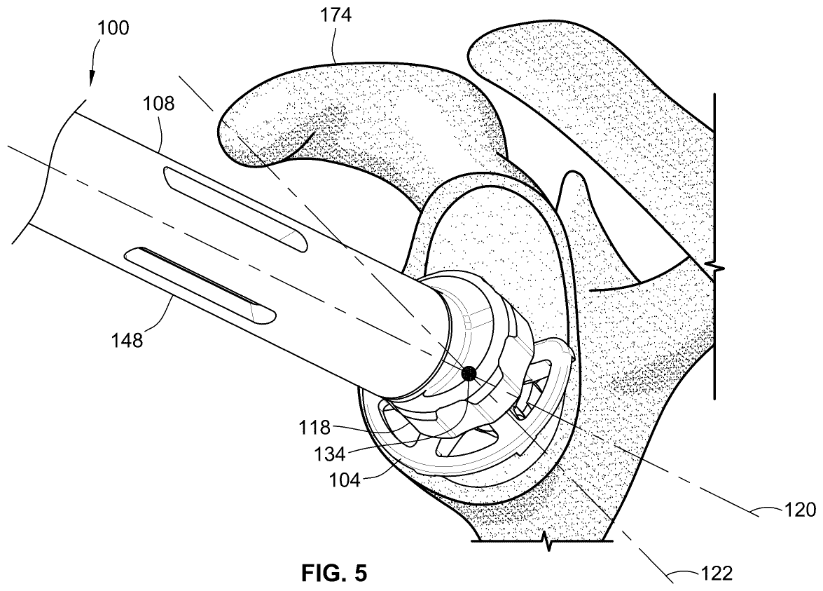

While the discussion of FIGS. 1-4 provide additional detail regarding a reamer assembly, the discussion of FIG. 5 describes the use of such a reamer assembly.

FIG. 5 is a perspective view of a reamer assembly 100 in use on a patient, according to some implementations of the present disclosure. As depicted in FIG. 5, the reamer assembly 100 is being used to ream bone in a patient's shoulder 174 (e.g., to remove bone from the patient's glenoid cavity). It should be noted that while FIG. 5 depicts the reamer assembly 100 is in use for a patient's shoulder, implementations are not so limited. That is, the reamer assembly 100 can be used for any suitable joint/bone.

The reamer assembly 100 includes a housing 102 and a reamer 104. The housing is oriented along the longitudinal axis 120. The lower housing 114 is angled relative to the upper housing 148. The reamer 104 is coupled to the lower housing 114. Because of the angled nature of the lower housing 114, the reamer 104 rotates about an axis that is different than the longitudinal axis. Specifically, as depicted in FIG. 5, the reamer 104 rotates about a second longitudinal axis 122.

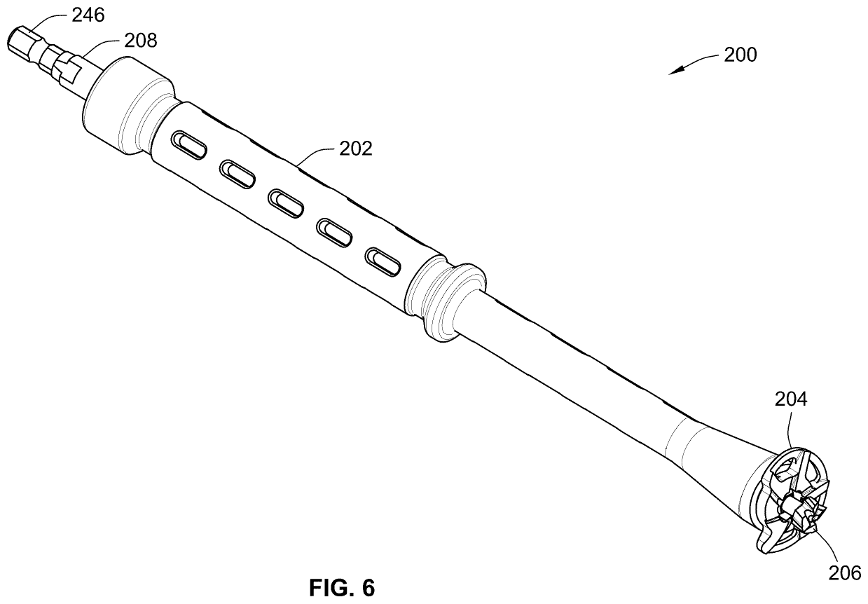

While the discussion of FIGS. 1-5 describes a first implementation of the present disclosure, the discussion of FIGS. 6-11 describes a second implementation of the present disclosure. While in the first implementation of the reamer assembly the housing includes an upper housing and a lower housing, in the second implementation of the reamer assembly the housing is a monolithic structure. It should be noted that, in some implementations, features of the first implementation of the reamer assembly and features of the second implementation of the reamer assembly can be combined, substituted, removed, added, etc. as desired.

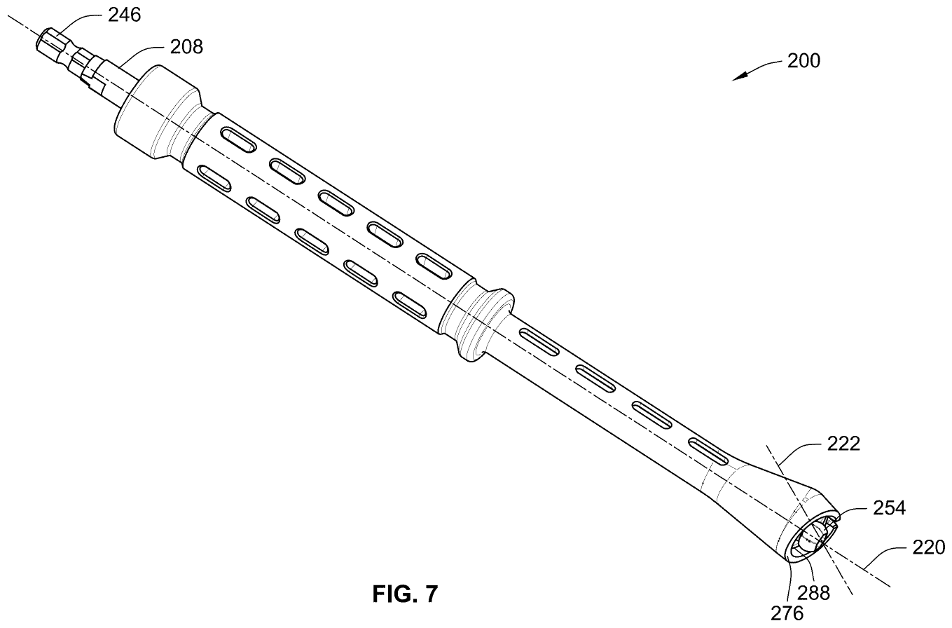

FIG. 6 is a perspective view of a second implementation of a reamer assembly 200, according to some implementations of the present disclosure. As depicted in FIG. 6, the reamer assembly 200 includes a housing 202, drive shaft 208, reamer 204, and boss drill 206. The housing 202 includes a channel extending therethrough. The drive shaft 208 is configured to be inserted into the housing 202 via the channel. Upon insertion, at least a portion of the drive shaft 208 is disposed within the housing 202. The drive shaft 208 is configured to rotate about a first rotational axis (i.e., the axis of rotation of the drive shaft 208). In some implementations, and as depicted in FIG. 6, a proximal end of the drive shaft 208 includes a mating mechanism 246. In such implementations, the mating mechanism 246 is configured to be engaged by, for example, a tool to impart rotational motion on the drive shaft 208. Ultimately, the rotation of the drive shaft 208 imparts rotational motion on the reamer 246 and the boss drill 206. As illustrated in FIG. 7, the first rotational axis (i.e., the axis of rotation of the drive shaft 208) is different than a second rotational axis (i.e., an axis of rotation of the reamer 204). While the reamer 204 rotates about the second rotational axis, the boss drill 206 rotates about the first rotational axis. As with the reamer 204, the drive shaft 208 imparts rotational motion on the boss drill 206.

FIG. 7 is a perspective view of a second implementation of a housing 202 and drive shaft 208 of a reamer assembly 200, according to some implementations of the present disclosure. Like the reamer assembly 200 depicted in FIG. 6, the reamer assembly 200 depicted in FIG. 7 includes a housing 202 and a drive shaft 208. The drive shaft 208 is at least partially seated within a channel of the housing 202. The channel of the housing 202 is aligned along a longitudinal axis 220. Because the drive shaft 208 is inserted into the housing 202 via the channel, the drive shaft 208 also rotates about the longitudinal axis. Accordingly, the axis of rotation of the drive shaft 208 is the same as the longitudinal axis.

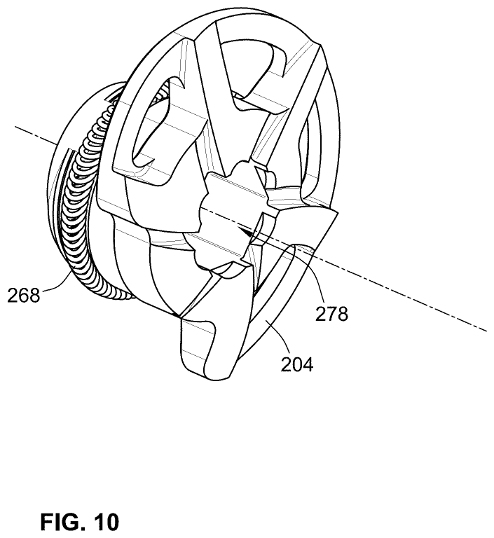

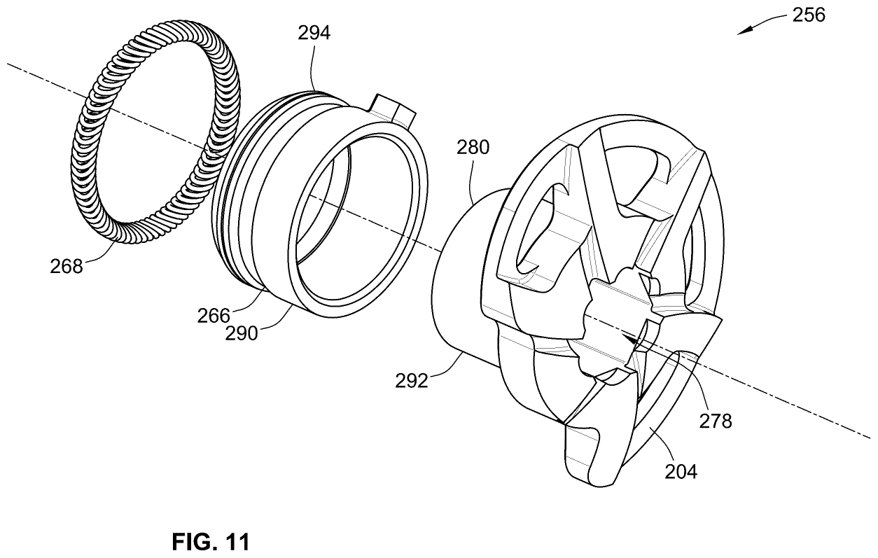

A lower portion of the housing 202 is angled relative to the longitudinal axis 220. For example, as depicted in FIG. 7, a distal surface 276 of the housing 222 is neither parallel nor orthogonal to the longitudinal axis 220. Rather, the distal surface 276 of the housing 202 is orthogonal to a second a second axis of rotation 222. Additionally, the housing 202 includes an interior surface 288. Like the distal surface 276, the interior surface 288 is neither parallel nor orthogonal to the longitudinal axis 220. Similar to the distal surface 276, the interior surface 288 is orthogonal to the second axis of rotation 222. The reamer 204 (not shown in FIG. 7) rides on the interior surface 288 of the housing 202. Because the reamer 204 rides on the interior surface 288 of the housing 202 and the interior surface 288 of the housing 202 is orthogonal to the second axis of rotation 222, the reamer 204, when imparted with rotational motion from the drive shaft 208, rotates about the second axis of rotation 222. Accordingly, the drive shaft 208, and boss drill if so equipped, rotate about the longitudinal axis 220 and the reamer 204 rotates about the second axis of rotation 222. In some implementations, a bearing (as shown in the example depicted in FIG. 11) seats on the interior surface 288 and, in use, is located between the interior surface 222 and the reamer.

An intersection of the longitudinal axis 220 and the second axis of rotation 222 can be located within, distal from, or proximal to the head of the reamer 204, as desired. In some implementations, the distal end of the drive shaft 208 includes a second mating mechanism 254. An internal mating mechanism of the reamer 204 is configured to mate with the second mating mechanism 254 to receive rotational motion from the drive shaft 208.

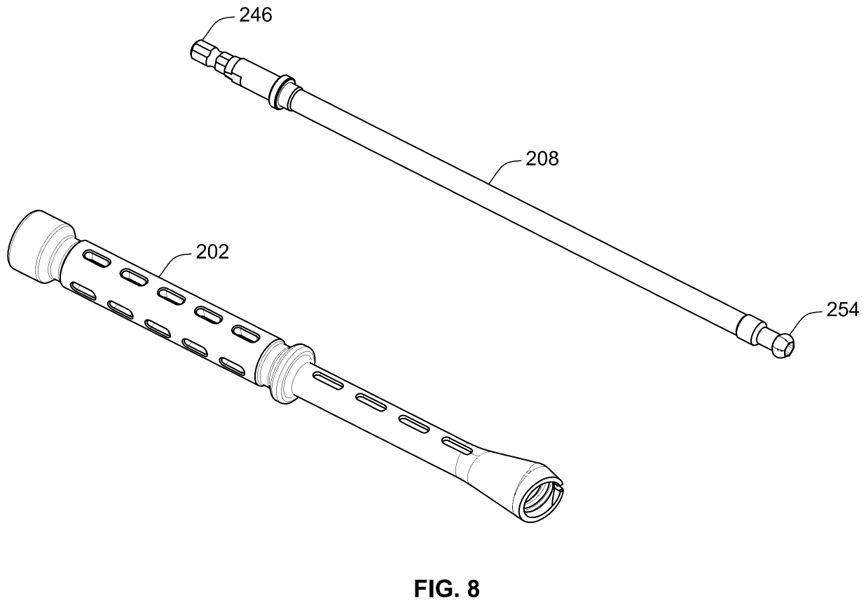

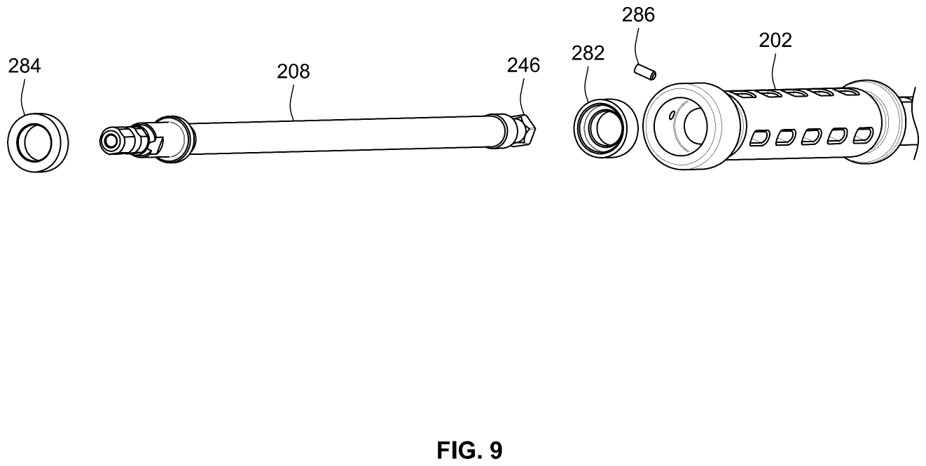

While the discussion of FIGS. 6 and 7 provides background regarding a second implementation of a reamer assembly according to the present disclosure, the discussion of FIGS. 8 and 9 provides additional detail regarding a housing and drive shaft of the reamer assembly according to some implementations.

FIGS. 8 and 9 are exploded views of a portion of a second implementation of a reamer assembly 200, according to some implementations of the present disclosure. FIGS. 8 and 11 include a housing 202, drive shaft 208, upper bearing 282, lower bearing 284, and retentive pin 286. The housing 202 includes a channel extending therethrough. The drive shaft 208 is configured to be at least partially inserted into the channel of the housing 202. The upper bearing 282 and lower bearing 284 provide a contact surface for the drive shaft 208. The retentive pin 286 is configured to be inserted into a bore of the housing. In some implementations, the bore and the retentive pin 286 are both threaded. The retentive pin 286 can be used to secure one or both of the upper bearing 282 and the drive shaft 208 within the housing 202. Though not shown in FIG. 9, the lower bearing 284 can be secured to the housing 202 in a similar manner via a second retentive pin. In one implementation, the lower bearing 284 can be seated against the interior surface of the housing 202 (depicted, for example, in FIG. 7). It should be noted that, allow shown as being secured to the housing 202 via the retentive pin 286, the upper bearing 282 (and lower bearing 284) can be secured to the housing 202 in any suitable manner. For example, the upper bearing 282 and/or lower bearing can be secured to the housing 202 via welds, an interference fit, clips, retentive members, fasteners, etc.

While the discussion of FIGS. 8 and 9 provides additional detail regarding a housing and drive shaft of the reamer assembly according to some implementations, the discussion of FIGS. 10 and 11 provides additional detail regarding a reamer of a reamer assembly according to some implementations.

FIG. 10 is a perspective view of a reamer 204 and FIG. 11 is an exploded view of the reamer 204, according to some implementations of the present disclosure. The reamer 204 is generally configured to remove bone. The reamer 204 is configured to rotate based on an interaction with a drive shaft. In some implementations, and as depicted in FIG. 10, the reamer 204 includes a through bore 278. The through bore 278 allows a boss drill to be at least partially inserted (e.g., a shank of the boss drill) into the reamer 204. In such implementations, a proximal end of the boss drill can mate with a portion of the drive shaft. Accordingly, the drive shaft can impart rotational motion on the reamer 204 and the boss drill.

Additionally, as shown in FIG. 10, in some implementations, the reamer 204 is configured to be secured within the housing via a third retentive member 268. Though the third retentive member 268 is depicted in FIG. 10 as being part of a larger assembly of the reamer 204, it should be noted that, in some implementations, the third retentive member 268 is located within the housing and the reamer 204 is inserted into the housing to secure the reamer 204 to the housing via the third retentive member 268. The third retentive member 268 can take any suitable form. For example, as depicted in the example shown in FIG. 10, the third retentive member 268 is a coil spring. In such implementations, the third retentive member 268 can seat, for example, in a retentive groove (e.g., a third retentive groove 266). As depicted in the example provided in FIG. 11, the third retentive groove 266 is located in a bushing 290. However, it should be noted that in addition to, or in lieu of, the third retentive groove 266 being located in the bushing 290, the third retentive groove (or an additional third retentive groove 266) can be located on an inner surface of the housing 202. The bushing 290 seats on a protrusion 292 of the reamer 204. In one implementation, a cap 294 is secured to a proximal end of the protrusion 292 (e.g., via welding, fasteners, etc.). In such implementations, the cap 294 can capture the bushing 290 on the protrusion 292 between the cap 294 and a head of the reamer 204. In such implementations, the bushing 290 can be secured to the cap 294 and/or the protrusions 292, or can be allowed to rotate relative to the cap 294 and/or the protrusion 292.

As discussed previously, the reamer 204 rides on a distal surface of the interior surface 288 of the housing 202. In some implementations, the reamer 204 and/or bearing assembly 212 include a mating surface 280. The mating surface 280 rides on the interior surface 288 of the housing 202. Additionally, as noted previously, in some implementations, the cap 294 is secured to the protrusion 292 of the reamer 204. In such implementations, at least a portion of the mating surface 280 and at least a portion of the cap 294 (e.g., a distal surface of the cap 294) can ride on the interior surface 288 of the housing 202.

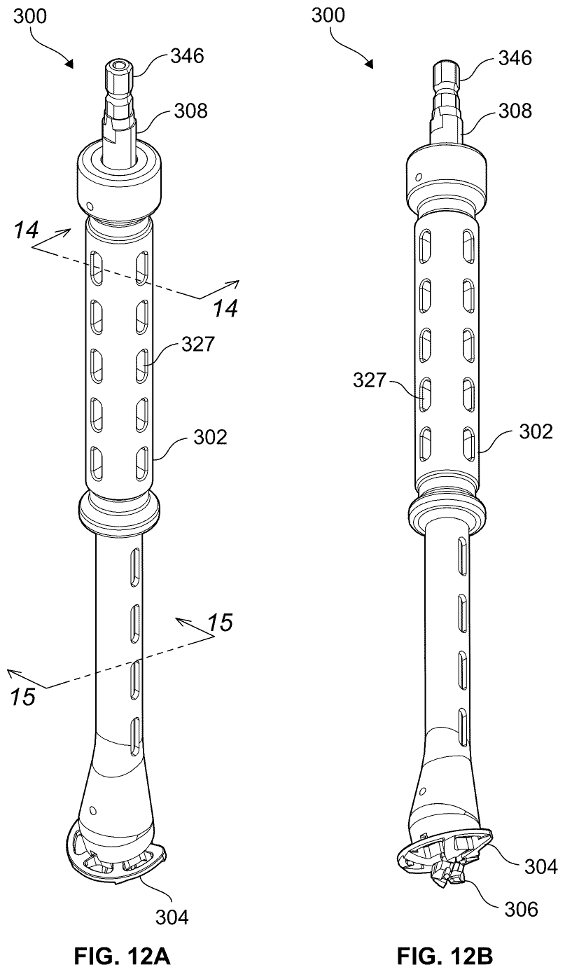

While the discussion of FIGS. 6-11 describes a second implementation of the present disclosure, the discussion of FIGS. 12-15 describes a third implementation of the present disclosure. Like the second implementation, the housing of the third implementation is a monolithic structure. It should be noted that, in some implementations, features of the first implementation of the reamer assembly, features of the second implementation of the reamer assembly, and features of the third implementation of the reamer assembly can be combined, substituted, removed, added, etc. as desired.

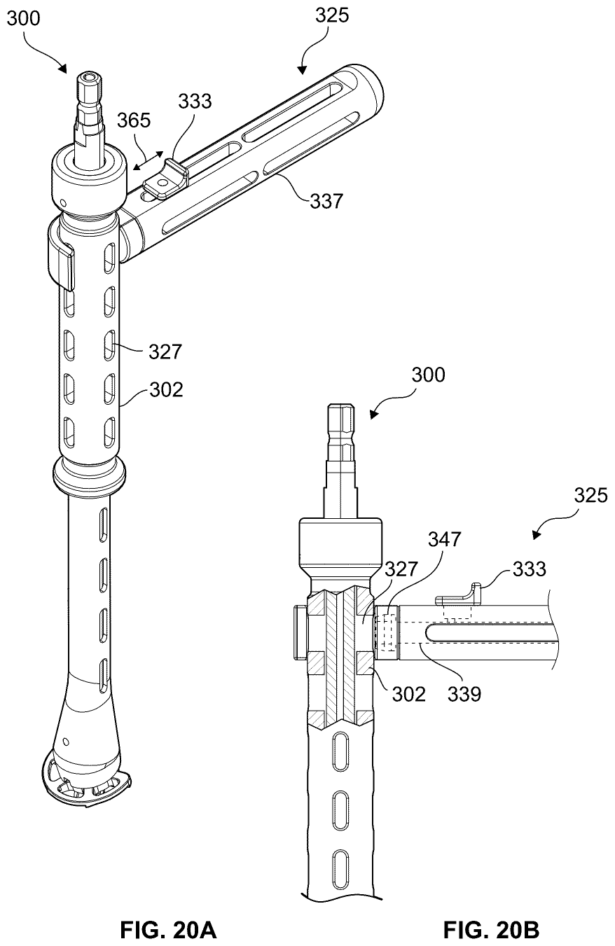

Referring to FIGS. 12A and 12B, a reamer assembly 300 is illustrated according to some implementations of the present disclosure. The reamer assembly 300 includes a housing 302, a drive shaft 308, a reamer 304, and a boss drill 306 (FIG. 12B). The housing 302, the drive shaft 308, the reamer 304, and the boss drill 306 are the same as, or similar to, the housing 102/202, the drive shaft 108/208, the reamer 104/204, and the boss drill 106/206 of the reamer assembly 100/200 illustrated in FIGS. 1-11. The drive shaft 308 is configured to be inserted into the housing 302 via the channel. Upon insertion, at least a portion of the drive shaft 308 is disposed within the housing 302. The drive shaft 308 is configured to rotate about a first rotational axis (e.g., a longitudinal axis of the housing 320). In some implementations, and as depicted in FIGS. 12A and 12B, a proximal end of the drive shaft 308 includes a mating mechanism 346. In such implementations, the mating mechanism 346 is configured to be engaged by, for example, a tool to impart rotational motion on the drive shaft 308. Ultimately, the rotation of the drive shaft 308 imparts rotational motion on the reamer 346 and the boss drill 306. The first rotational axis is different than a second rotational axis 322 about which the reamer 304 rotates. While the reamer 304 rotates about the second rotational axis, the boss drill 306 rotates about the first rotational axis. As with the reamer 304, the drive shaft 308 imparts rotational motion on the boss drill 306. As illustrated in FIG. 15, an intersection 334 of the first rotational axis (e.g., the longitudinal axis of the housing) and the second rotational axis 322 is located within the head of the reamer 304. As depicted in FIGS. 12A and 12B, the housing 302 includes a plurality of apertures 327. The apertures can function to dissipate heat and/or allow cleaning solution (e.g., an autoclave cleaning solution, steam, etc.) to flow through the housing 302. Additionally, in some implementations and as described with respect to FIGS. 20-23, the apertures 127 can be used to secure accessories to the housing 302 of the reamer assembly 300.

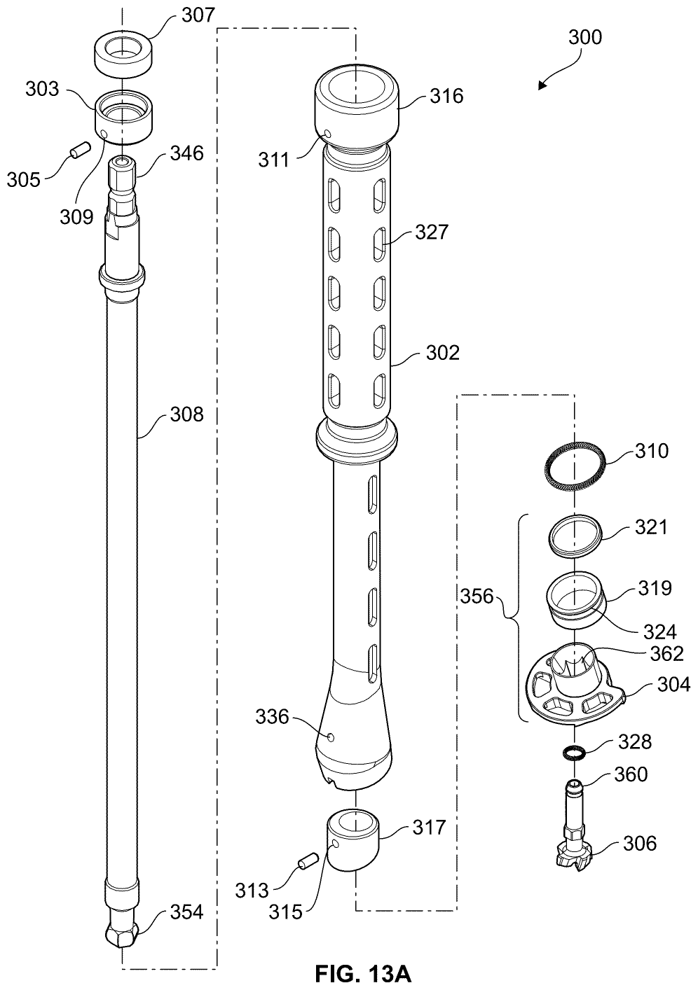

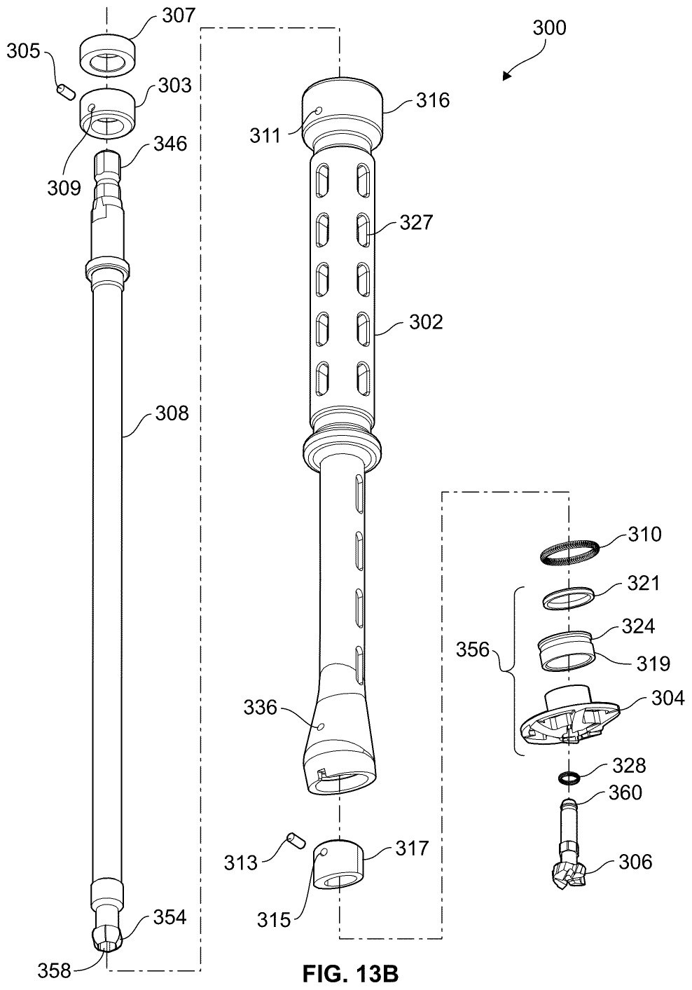

FIGS. 13A and 13B are exploded views of the reamer assembly 300 depicted in FIGS. 12A and 12B. In the example depicted in FIGS. 13A and 13B, the reamer assembly 300 generally includes an upper bushing 307, an upper insert 303, an upper pin 305, a drive shaft 308, a housing 302, a lower insert 317, a reaming head assembly 356, a boss drill 306, and a second retentive member 328. While FIGS. 13A and 13B depict each of the upper bushing 307, upper insert 303, upper pin 305, drive shaft 308, housing 302, lower insert 317, reaming head assembly 356, boss drill 306, and second retentive member 328, it should be noted that the reamer assembly 300 can include greater, or fewer, components and the components depicted in FIGS. 13A and 13B are but an example for illustrative purposes

As indicated in FIGS. 13A and 13B, the upper bushing 307 seats in the upper insert 303. Movement of the upper bushing 307 is prevented or limited relative to the upper insert 303 via an upper insert pin 305. The upper insert 303 includes a bore 309, and the upper insert pin 305 extends through the bore 309 to contact the upper bushing 307. For example, the upper insert pin 305 can function as a set screw, or proceed into a receiving bore in the upper bushing 307. The upper insert 303 seats in an upper collar 316 portion of the housing 302. As depicted in FIGS. 13A and 13B, the upper collar 316 portion of the housing can include a bore 311. The upper insert pin 305 extends through the bore 311 of the upper collar 316 portion of the housing 302 and into the bore 309 of the upper insert 303 to prevent or limit rotational motion of the upper insert 303 relative to the upper collar 316 portion of the housing 302. The lower insert 317 seats within a distal end of the housing 302. The lower insert 317 includes a bore 313 through which a lower insert pin 313 is inserted. Additionally, the housing 302 includes a bore 321. The lower insert pin 313 prevents or limits motion of the lower insert 317 relative to the housing 302.

The reaming head assembly 356 generally includes a first retentive member 310, a cap 321, a reamer bushing 319, and the reamer 304. When assembled, the reamer bushing 319 and the cap 321 seat on the reamer 304. The first retentive member 310 seats in a first retentive groove 324. As shown in FIG. 15, the housing 302 includes a corresponding first retentive groove 324. The first retentive member 310 secures the reamer head assembly 356 to the housing 302 via the first retentive groove 324. The boss drill 306, when the reamer assembly 300 is so equipped, is inserted through the reamer 304 and secured to the drive shaft 308 via an internal mating mechanism 358 of the drive shaft, as described in more detail with respect to FIG. 15. The drive shaft 308 includes a second mating mechanism 358. The drive shaft's 308 second mating mechanism 358 interfaces with an internal mating mechanism 362 of the reamer 304 to impart rotational motion on the reamer 304. As with the first and second implementations of the reamer assembly, the drive shaft 308 of the third implementation of the reamer assembly 300 rotates about a first rotaional axis, which is coincident with a longitudinal axis of the housing 302. Similarly, the lower portion of the housing 302 is angled relative to the rest of the housing 302, such that the reamer 304 is not perpendicular to the longitudinal axis when installed on the housing 302. As with the first and second implementations of the reamer assembly, the reamer 304 rotates about a second rotational axis that is neither parallel to nor coincident with the longitudinal axis. Similarly, in some implementations, the intersection of the longitudinal axis and the second rotational axis is within the reamer 304.

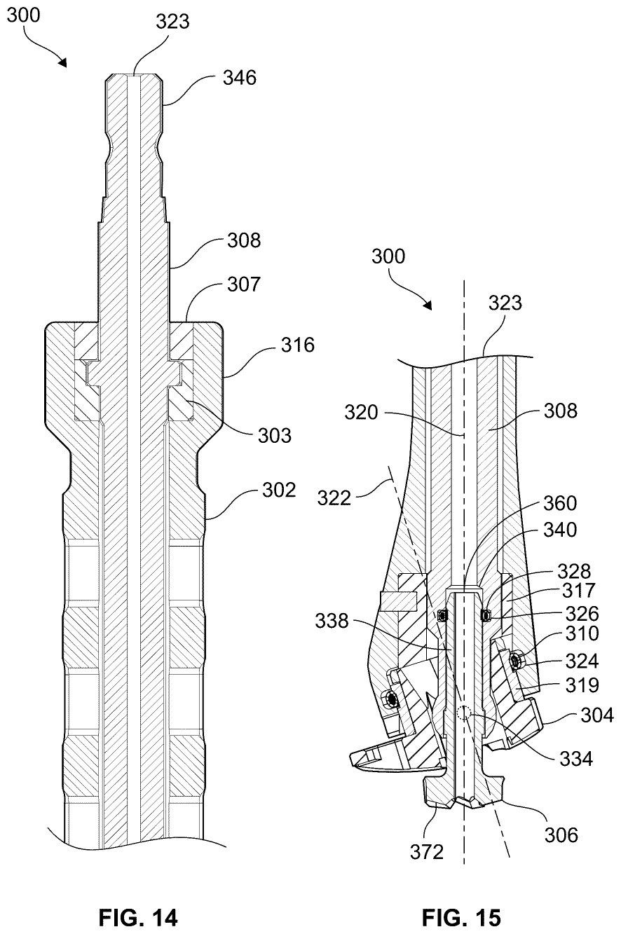

While the discussion of FIGS. 12A-12B and 13A-13B provides detail regarding the reamer assembly 300, the discussion of FIGS. 14 and 15 provides additional detail regarding the coupling of various components of the reamer assembly 300.

FIGS. 14 and 15 are sectional views of portions of the reamer assembly 300, taken along lines 14-14 and 15-15 of FIG. 12A, respectively, according to some implementations of the present disclosure. As previously noted and depicted in FIGS. 14 and 15, the reamer assembly 300 includes the housing 302, the upper bushing 307, the upper insert 303, the drive shaft 308, the reamer 304, and the boss drill 306. The drive shaft 308 is at least partially inserted into the housing 302 and is configured to rotate within the housing 302. In some implementations, the drive shaft 308 is cannulated, and includes a channel 323 extending therethrough. The channel 323 is configured to receive a guide wire (e.g., a K-wire, pin, etc.). The upper insert 303 seats inside the upper collar 316 portion of the housing 302. The upper bushing 307 seats inside the upper collar 316 portion of the housing 302 proximal from the upper insert 303.

The reaming head 304 and the boss drill 306 (if equipped) are secured to the drive shaft 308 and/or housing 302. When the reamer 304 is secured to the drive shaft 308 and/or housing 302, the reamer bushing 319 is located is located between the reamer 304 and an interior surface of the housing 302 and/or lower insert 317. The reamer bushing 319 and/or housing 302 include a first retentive groove 324 (e.g., one of the two components can include a groove, both of the components can include a groove, both of the components can include a portion of a groove, etc.). The first retentive member 310 seats in the first retentive groove 324, aiding in securing the reamer 304, reaming head assembly 356, and/or reamer bushing 319 to the housing 302. The cap 321 is secured to the reamer bushing 319. Similarly, a second retentive member 328 can be used to secure the boss drill 306 to the drive shaft 308. As depicted in FIG. 15, the boss drill 306 and/or the drive shaft 308 can include a second retentive groove 326 (e.g., one of the two components can include a groove, both of the components can include a groove, both of the components can include a portion of a groove, etc.). The second retentive member 328 seats in the second retentive groove 326, aiding in securing the boss drill 306 to the drive shaft 308.

In some implementations of the present disclosure, in addition to or in lieu of the second retentive member 328 and the second retentive groove 326, the drive shaft 308 and/or boss drill 306 can include features to aid in securing and/or locating the boss drill 306 to/within the drive shaft 308. For example, in some implementations, the boss drill 306 and/or drive shaft 308 can be dimensioned to limit and/or control the depth of the boss drill 306 in the drive shaft 308. In the example depicted in FIG. 15, an inner diameter of the drive shaft 308 is not uniform. Rather, the inner surface of the drive shaft 308 includes a ledge 340. In the proximal direction from the ledge 340, the inner diameter of the drive shaft 308 is smaller than the inner diameter of the drive shaft 308 in the distal direction from the ledge 340. The boss drill includes a shank 338 and a cutting element 372. The cutting element 372 is located at the distal end of the shank 338. The shank 338 has a diameter such that it can be inserted within the drive shaft 308. However, the diameter of the shank 338 is greater than an inner diameter of the drive shaft 308 proximal of the ledge 340. Accordingly, in the example depicted in FIG. 15, the ledge 340 acts as a physical stop that prevents the boss drill 306 from being inserted too deeply into the drive shaft 308. That is, the shank 338 of the boss drill 306 will physically contact the ledge 340 to limit the depth at which the boss drill 306 can be inserted into the drive shaft.

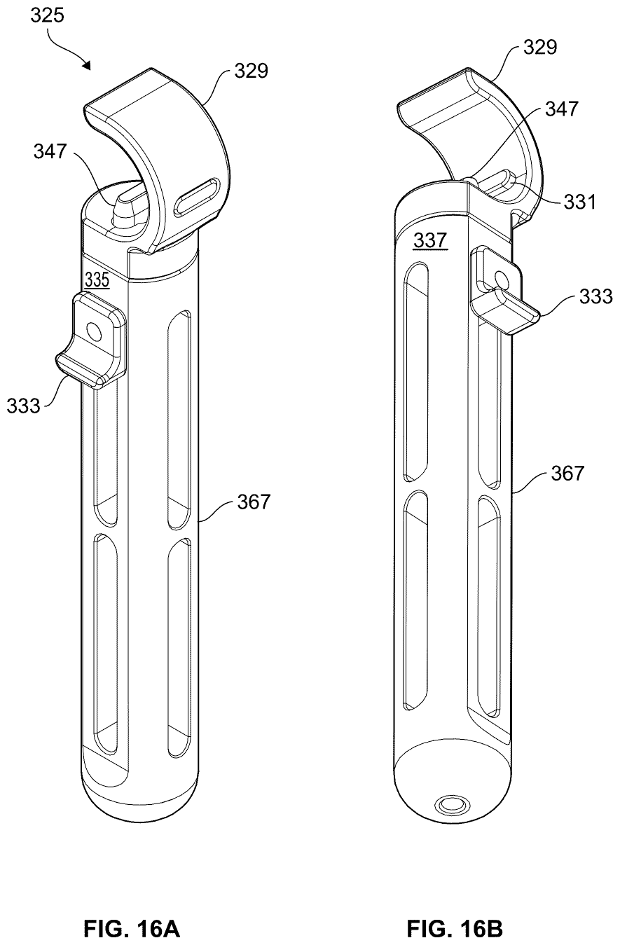

While the discussion of FIGS. 12A-15 describes a reamer assembly, the discussion of FIGS. 16-23 describes a handle that can be used with the reamer assembly 100, reamer assembly 200, and/or reamer assembly 300.

FIGS. 16A and 16B are perspective views of a handle 325 for use with a reamer assembly, according to some implementations. The handle 325 is configured to be secured to a reamer assembly to aid a user with manipulation and use of the reamer assembly. It should be noted that though the handle 352 is depicted as being used with the reamer assembly 300 of FIGS, 12A-15, implementations are not so limited. That is, the handle 352 can be adapted for use with any of the reamer assemblies depicted and described herein and/or any of the implementations of the reamer assemblies can be adapted for use with the handle 352.

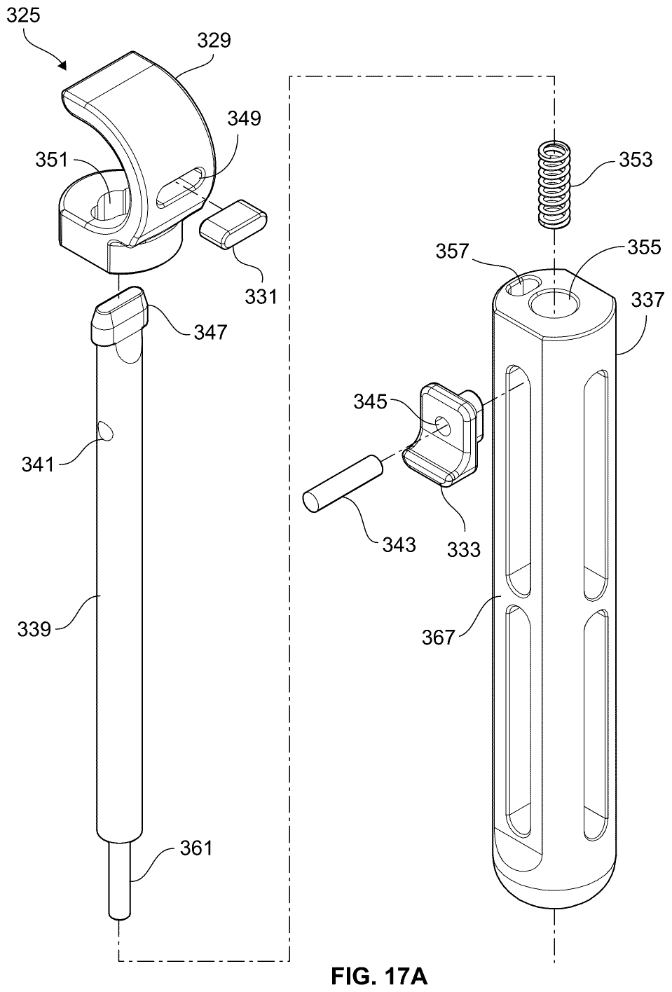

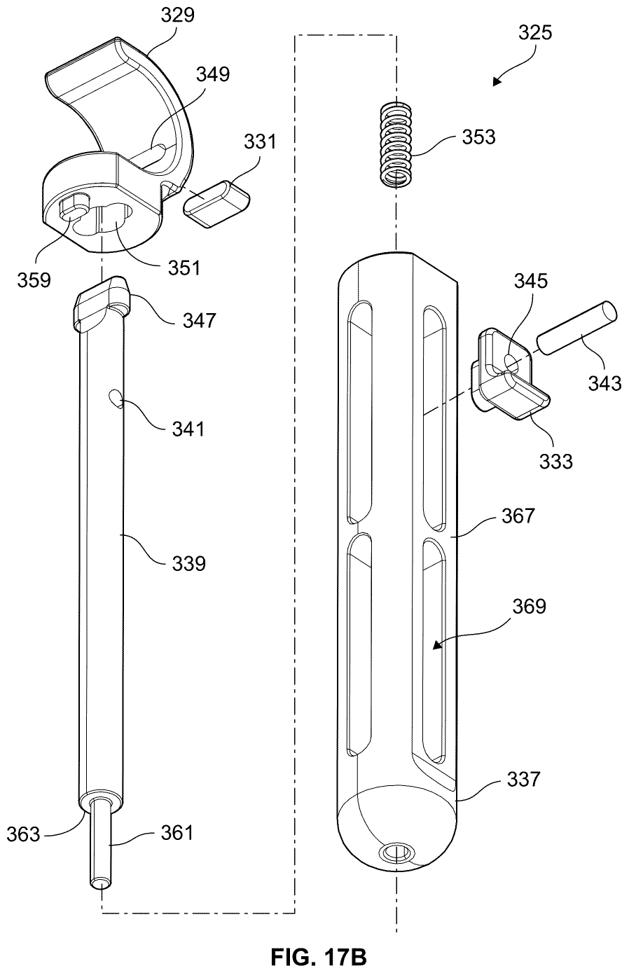

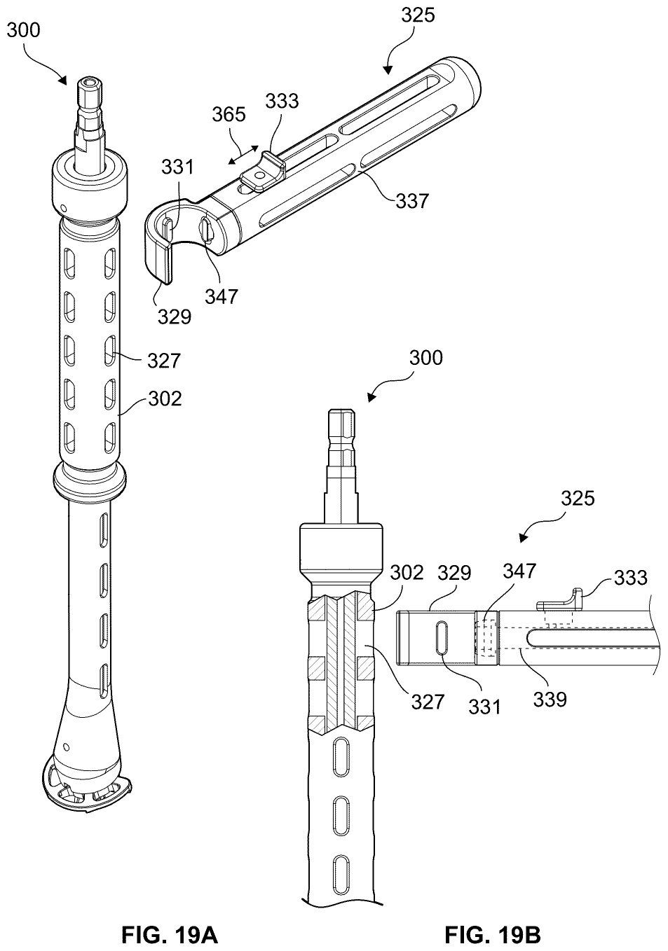

The handle 352 generally includes a body 337, a lever 333, and a movable protrusion 347. The lever 333 is coupled to the movable protrusion 347 to allow a user to manipulate the movable protrusion 347 between an extended position (e.g., as shown in FIGS. 16A-16B, 18A-18B, and 21A-21B) and a retracted position (e.g., as shown in FIGS. 91A-19B and 20A-20B). In the extended position, the movable protrusion 347 is configured to interface with an aperture 327 of the reamer assembly to aid in securing the handle 325 to the reamer assembly. The body 337 includes a connection portion 329 that extends from the body 337. The connection portion is configured to extend around at least a portion of a housing of a reamer assembly. In the example depicted in FIGS. 16A-16B, the connection portion 329 is generally semicircular, as it is designed to interface with a reamer assembly including a generally cylindrical housing. The connection portion 329 includes a protrusion 331 extending therefrom. In the example depicted in FIGS. 17A and 17B, the protrusion 331 seats in a connection portion aperture 349. The protrusion 331 is configured to interface with an aperture 327 of the reamer assembly to aid in securing the handle 325 to the reamer assembly.

FIGS. 17A and 17B are exploded views of the handle 325, according to some implementations. The handle 325 generally includes the body 337, the movable protrusion 347, a rod 339, the connection portion 329, the protrusion 331, the lever 333, a fastener 343, and a biasing member 353. The movable protrusion 347 is located at the end of the rod 339, and the rod is inserted into a channel 355 of the body 337. The rod 339 includes an end portion 361 which is opposite the movable protrusion 347. A diameter of the end portion 361 is smaller than a diameter of other portions of the rod 339, creating a ledge 363. The biasing spring 353 seats over the end portion 361 of and interfaces with the ledge 363 and a bottom of the body 337 to bias the movable protrusion 347 in the extended position. When in the extend position, the movable protrusion 347 extends through an opening 351 of the connection portion 329.

Though it can be, in the example depicted in FIGS. 17A and 17B, the connection portion 329 is not monolithically formed with the body 337. The body 337 includes a receptacle 357. The connection portion 329 includes an extension 359 protruding from the connection portion 329. When assembled, the extension 359 seats in the receptacle 357 to aid in locating the connection portion 329 on the body 337 and/or prevent movement of the connection portion 329 relative to the body 337.

The lever 333 is coupled to the rod 339 via a fastener 343. The fastener 343 can be, for example a screw, pin, rivet, etc. The fastener 343 extends through a lever aperture 345 of the lever 333 and into an aperture 341 in the rod 339, coupling the lever 333 to the rod 339. The user manipulates the lever 333 to move the movable protrusion 347 between the extended and retracted positions. As shown in the example depicted in FIGS. 17A and 17B, the body 337 has a generally round cross section with a flat surface 367. Though this is not required, in the example depicted in FIGS. 17A and 17B, the lever 333 is located within the flat portion 367. Additionally, the body 337 includes a plurality of elongated apertures 369. The lever 333 is secured to the rod 339 through one of the plurality of elongated apertures 369.

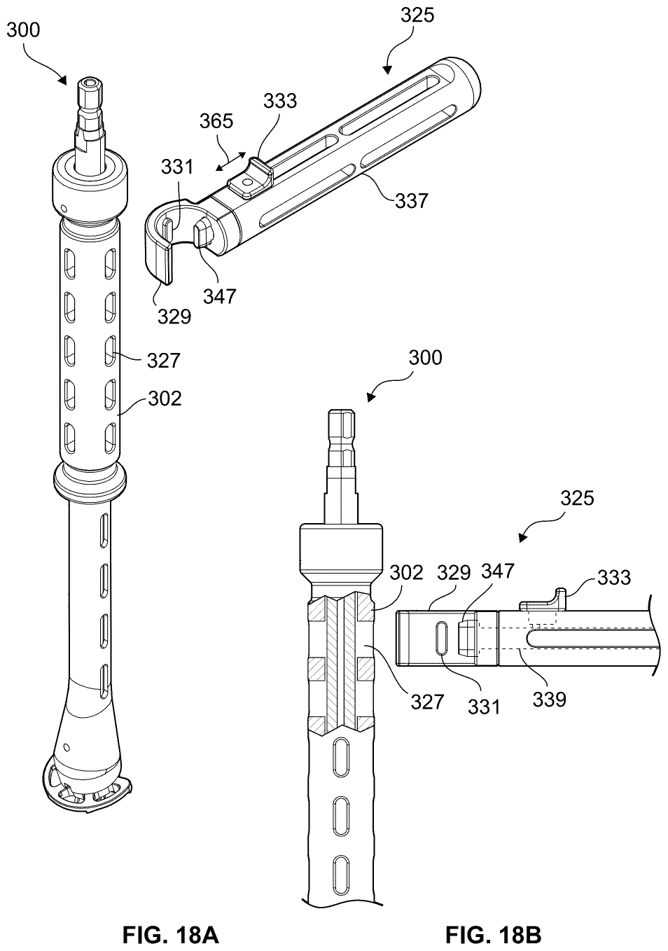

While the discussion of FIGS. 16A-17B provides additional detail regarding the handle, the discussion of FIGS. 18A-21B describes the use of the handle with a reamer assembly.

FIGS. 18A-21B generally depict securing the handle 337 to the reamer assembly 300. In FIGS. 18A and 18B, the handle 325 has not been secured to the reamer assembly 300 and the movable protrusion 347 is in the extended position. To secure the handle 337 to the reamer assembly 300, the user manipulates the lever 333, as depicted by an arrow 365, to move the movable protrusion 347 from the extended position (in which the movable protrusion 347 extends beyond the body 337 of the handle 325) to the retracted position (in which the movable protrusion 347 is located within the body 337 of the handle 325). In FIGS. 19A and 19B, the handle 325 has not been secured to the reamer assembly and the movable protrusion 347 is in the retracted position. That is, FIGS. 19A and 19B depict the handle 325 after the user has manipulated the lever such that the movable protrusion has moved from the extended position to the retracted position.

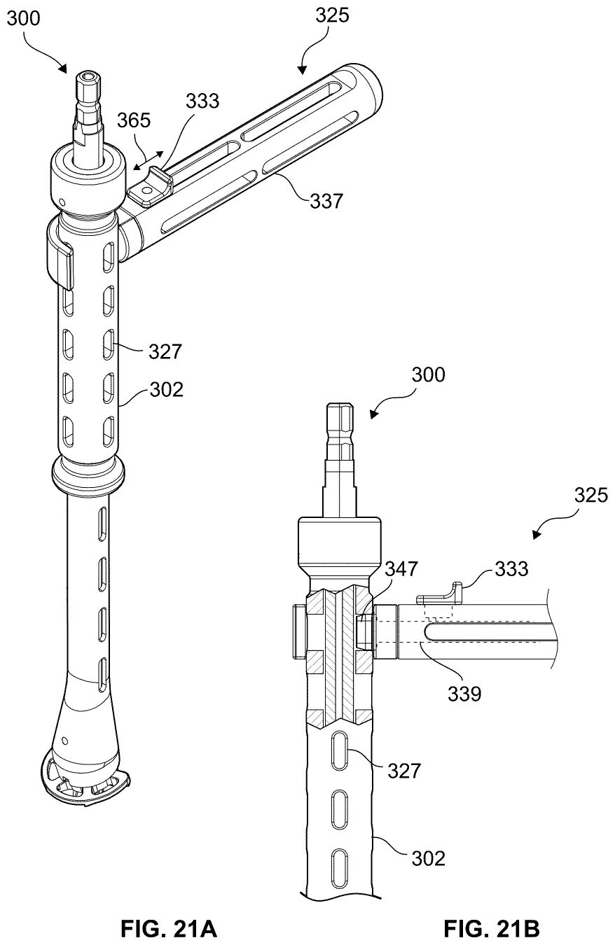

In FIGS. 20A and 20B, the handle 325 has been placed on the reamer assembly 300, but the movable protrusion 347 is still in the retracted position of FIGS. 19A and 19B. The handle 325 interfaces with the reamer assembly 300 via the connection portion 329. That is, the connection portion 329 extends at least partially around the housing 302 of the reamer assembly 300. In FIGS. 21A and 21B, the user has manipulated the lever 333, as indicated by the arrow 356, such that the movable protrusion 347 is in the extended position. When the handle is secured to the reamer assembly 300, as shown in FIGS. 21A and 21B, the movable protrusion seats in an aperture 327 of the housing 302 of the reamer assembly. Similarly, the protrusion 331 seats in another one of the apertures 327 of the housing 302 of the reamer assembly 300. The housing 302 of the reamer assembly 300 includes a plurality of apertures 327. The apertures are arranged about the housing 302 to allow the user to secure the handle 325 to the reamer assembly 300 in a variety of positions and/or orientations.

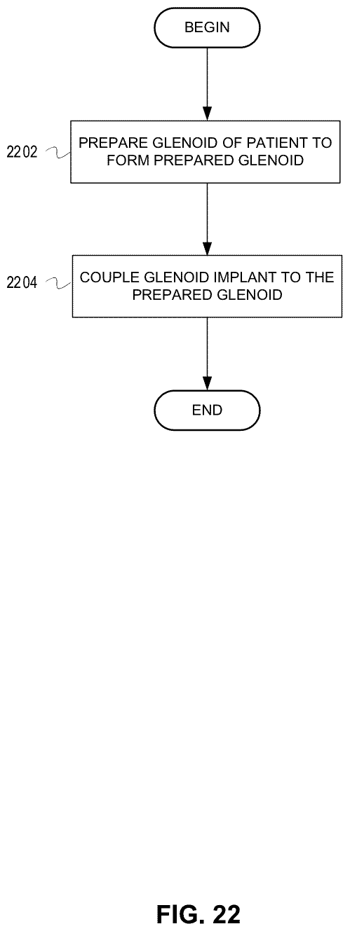

While the discussion of FIGS. 1-21B describes examples of reamer assemblies and handles for use with reamer assemblies, the discussion of FIG. 22 describes a method of coupling a glenoid implant.

FIG. 22 is a flowchart depicting example operations for coupling a glenoid implant, according to some implementations. The flow beings at block 2202.

At block 2202, a glenoid of a patient is prepared to form a prepared glenoid. For example, the glenoid of the patient can be prepared using a reamer assembly, such as those described herein. Generally, the reamer assembly is used to ream or otherwise remove bone from the patient's glenoid. Additionally, in some implementations, a boss drill can be used with the reamer assembly to drill one or more central and/or peripheral bores in or to form the prepared glenoid. Further, a separate drill can be used to drill the central and/or peripheral bores. The prepared glenoid can have a flat and/or curved surface. The flow continues at block 2204.

At block 2204, a glenoid implant is coupled to the prepared glenoid. The glenoid implant can be coupled to the prepared glenoid in any suitable manner. For example, the glenoid implant can be coupled to the prepared glenoid via fasteners, such as threaded fasteners, pegs, posts, clips, etc. The glenoid implant has a bone-facing surface. The bone-facing surface can take any suitable shape, and be flat and/or curved. In some implementations, the prepared glenoid can have a surface that complements the shape/surface of the glenoid implant.

### Implementations

Implementation 1: A reamer assembly comprising: a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, a boss drill configured to be removably coupled to the drive shaft such that the drive shaft is configured to cause the boss drill to rotate about the first axis of rotation.

Implementation 2: The reamer assembly of implementation 1, wherein an intersection of the first axis of rotation and the second axis of rotation is located within the reamer.

Implementation 3: The reamer assembly of implementation 1 or implementation 2, wherein a proximal end of the drive shaft is configured to be coupled to a driving tool, wherein a distal end of the drive shaft includes a ball hex, wherein the reamer includes an internal hex, and wherein the reamer is configured to mate with the drive shaft via the ball hex and the internal hex.

Implementation 4: The reamer assembly of any one of implementation 1 to implementation 3, wherein the second axis of rotation is fixed for the reamer assembly.

Implementation 5: The reamer assembly of any one of implementation 1 to implementation 4, wherein a distal portion of the housing includes a recess, and further comprising: a retentive member, wherein the retentive member seats within the recess, and wherein the retentive member is configured to secure the reamer within the housing.

Implementation 6: The reamer assembly of implementation 5, wherein the retentive member is one or more of a spring, a clip, a ring, and a fastener.

Implementation 7: The reamer assembly of any one of implementation 1 to implementation 6, wherein the boss drill is configured to be inserted into the drive shaft, wherein the drive shaft includes a recess, and further comprising: a retentive member, wherein the retentive member seats within the recess, and wherein the retentive member is configured to secure the boss drill within the drive shaft.

Implementation 8: The reamer assembly of any one of implementation 1 to implementation 7, wherein the drive shaft is cannulated.

Implementation 9: The reamer assembly of implementation 8, further comprising: a guide wire, wherein the guide wire is configured to be inserted through the drive shaft.

Implementation 10: The reamer assembly of any one of implementation 1 to implementation 9, wherein a distal portion of the housing is at angle with respect to a proximal portion of the housing, and wherein the angle provides the rotational axis of the reamer.

Implementation 11: The reamer assembly of any one of implementation 1 to implementation 10, wherein the first axis of rotation is coincident with the longitudinal axis.

Implementation 12: The reamer assembly of any one of implementation 1 to implementation 11, wherein a maximum diameter of the reamer is greater than a maximum diameter of the boss drill.

Implementation 13: The reamer assembly of any one of implementation 1 to implementation 12, wherein the boss drill includes a shank and a cutting element, wherein the cutting element is located at a distal end of the shank, and wherein the shank includes a mating mechanism.

Implementation 14: The reamer assembly of any one of implementation 1 to implementation 13, wherein an inner surface of the drive shaft includes a ledge, and wherein a shank of the drive shaft seats on the ledge when the boss drill is coupled to the drive shaft to limit a depth into the drive shaft which the shank can extend.

Implementation 15: A reamer assembly comprising: a housing forming a channel extending therethrough and defining a longitudinal axis, a drive shaft at least partially positioned in the channel and configured to rotate about a first axis of rotation, and a reamer configured to be removably coupled to the housing such that the drive shaft (i) engages the reamer and (ii) is configured to cause the reamer to rotate about a second axis of rotation that is different than the first axis of rotation, and wherein an intersection of the first axis of rotation and the second axis of rotation is located within the reamer.

Implementation 16: The reamer assembly of implementation 15, further comprising: a boss drill configured to be removably coupled to the drive shaft such that the drive shaft is configured to cause the boss drill to rotate about the first axis of rotation.

Implementation 17: The reamer assembly of implementation 15 or implementation 16, wherein a proximal end of the drive shaft is configured to be coupled to a driving tool, wherein a distal end of the drive shaft includes a ball hex, wherein the reamer includes an internal hex, and wherein the reamer is configured to mate with the drive shaft via the ball hex and the internal hex.

Implementation 18: The reamer assembly of any one of implementation 15 to implementation 17, wherein the second axis of rotation is fixed for the reamer assembly.

Implementation 19: The reamer assembly of any one of implementation 15 to implementation 18, wherein a distal portion of the housing includes a recess, and further comprising: a retentive member, wherein the retentive member seats within the recess, and wherein the retentive member is configured to secure the reamer within the housing.

Implementation 20: The reamer assembly of implementation 19, wherein the retentive member is one or more of a spring, a clip, a ring, and a fastener.

Implementation 21: The reamer assembly of any one of implementation 15 to implementation 20, wherein the boss drill is configured to be inserted into the drive shaft, wherein the drive shaft includes a recess, and further comprising: a retentive member, wherein the retentive member seats within the recess, and wherein the retentive member is configured to secure the boss drill within the drive shaft.

Implementation 22: The reamer assembly of any one of implementation 15 to implementation 21 wherein the drive shaft is cannulated.

Implementation 23: The reamer assembly of implementation 22, further comprising: a guide wire, wherein the guide wire is configured to be inserted through the drive shaft.

Implementation 24: The reamer assembly of any one of implementation 15 to implementation 23, wherein a distal portion of the housing is at angle with respect to a proximal portion of the housing, and wherein the angle provides the rotational axis of the reamer.

Implementation 25: The reamer assembly of any one of implementation 15 to implementation 24, wherein the first axis of rotation is coincident with the longitudinal axis.

Implementation 26: The reamer assembly of any one of implementation 15 to implementation 25 wherein a maximum diameter of the reamer is greater than a maximum diameter of the boss drill.

Implementation 27: The reamer assembly of any one of implementation 15 to implementation 26 wherein the boss drill includes a shank and a cutting element, wherein the cutting element is located at a distal end of the shank, and wherein the shank includes a mating mechanism.

Implementation 28: The reamer assembly of any one of implementation 15 to implementation 27, wherein an inner surface of the drive shaft includes a ledge, and wherein a shank of the drive shaft seats on the ledge when the boss drill is coupled to the drive shaft to limit a depth into the drive shaft which the shank can extend.

Implementation 29: A handle for use with a reamer assembly, the handle comprising: a body, a movable protrusion movably coupled to the body and configured to be moved between an extended position in which the movable protrusion extends beyond the body and a retracted position, a lever coupled to the movable protrusion and configured to move the movable protrusion between the extended position and the retracted portion, and a connection portion configured to interface with a reamer assembly.

Implementation 30: The handle of implementation 29, further comprising: a rod located within a channel of the body, wherein the movable protrusion is secured to the rod and a biasing member configured to bias the rod such that the movable protrusion is biased in the extended position.

Implementation 31: The handle of implementation 30, wherein the rod includes an end portion having a smaller diameter than an upper portion of the rod, wherein the biasing member seats on the end portion.

Implementation 32: The handle of implementation 30 or implementation 31, wherein the biasing member is spring.

Implementation 33: The handle of any one of implementation 30 to implementation 32, wherein the rod includes an aperture and the lever includes a lever aperture, and further comprising: a pin configured to couple the lever to the rod via the aperture and the lever aperture.

Implementation 34: The handle of any one of implementation 29 to implementation 33, wherein the connection portion includes a protrusion, and wherein the movable protrusion and the protrusion are configured to seat in apertures of the reamer to secure the handle to the reamer assembly.

Implementation 35: The handle of any one of implementation 29 to implementation 34, wherein the body includes a least one flat surface, and wherein the lever is located on one of the at least one flat surfaces.

Implementation 36: The handle of any one of implementation 29 to implementation 35, wherein the body includes a plurality of elongated apertures, and wherein the lever is coupled to the movable protrusion through one of the plurality of apertures.

Implementation 37: The handle of any one of implementation 29 to implementation 36, wherein the body and the connection portion are monolithically formed.

Implementation 38: The handle of any one of implementation 29 to implementation 37, wherein the connection portion is semicircular.

Implementation 39: The handle of any on of implementation 39 to implementation 38, wherein the connection portion includes an opening, and wherein the movable protrusion is configured to extend through the opening.

Implementation 40: A reaming system comprising: a reamer assembly comprising: a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, a boss drill configured to be removably coupled to the drive shaft such that the drive shaft is configured to cause the boss drill to rotate about the first axis of rotation, and a handle for use with the reamer assembly comprising: a body, a movable protrusion movably coupled to the body and configured to be moved between an extended position in which the movable protrusion extends beyond the body and a retracted position, a lever coupled to the movable protrusion and configured to move the movable protrusion between the extended position and the retracted portion, and a connection portion configured to interface with a reamer assembly.

Implementation 41: The reaming system of implementation 40, wherein the handle further comprises: a rod located within a channel of the body, wherein the movable protrusion is secured to the rod and a biasing member configured to bias the rod such that the movable protrusion is biased in the extended position

Implementation 42: The reaming system of implementation 41, wherein the rod includes an end portion having a smaller diameter than an upper portion of the rod, wherein the biasing member seats on the end portion.

Implementation 43: The reaming system of implementation 41 or implementation 42, wherein the biasing member is a spring.

Implementation 44: The reaming system of any one of implementation 41 to implementation 43, wherein the rod includes an aperture and the lever includes a lever aperture, and further comprising: a pin configured to couple the lever to the rod via the aperture and the lever aperture.

Implementation 45: The reaming system of any one of implementation 40 to implementation 44, wherein the connection portion includes a protrusion, and wherein the movable protrusion and the protrusion are configured to seat in apertures of the reamer assembly to secure the handle to the reamer assembly.

Implementation 46: The reaming system of any one of implementation 40 to implementation 45, wherein the body includes a least one flat surface, and wherein the lever is located on one of the at least one flat surfaces.

Implementation 47: The reaming system of any one of implementation 40 to implementation 46, wherein the body includes a plurality of elongated apertures, and wherein the lever is coupled to the movable protrusion through one of the plurality of apertures.

Implementation 48: The reaming system of any one of implementation 40 to implementation 47, wherein the body and the connection portion are monolithically formed.

Implementation 49: The reaming system of any one of implementation 40 to implementation 48, wherein the connection portion is semicircular.

Implementation 50: The reaming system of any one of implementation 40 to implementation 49, wherein the connection portion includes an opening, and wherein the movable protrusion is configured to extend through the opening

Implementation 51: The reaming system of any one of implementation 40 to implementation 50, wherein an intersection of the first axis of rotation and the second axis of rotation is located within the reamer.

Implementation 52: The reaming system of any one of implementation 40 to implementation 51, wherein a proximal end of the drive shaft is configured to be coupled to a driving tool, wherein a distal end of the drive shaft includes a ball hex, wherein the reamer includes an internal hex, and wherein the reamer is configured to mate with the drive shaft via the ball hex and the internal hex.

Implementation 53: The reaming system of any one of implementation 40 to implementation 52, wherein the second axis of rotation is fixed for the reamer assembly.

Implementation 54: The reaming system of any one of implementation 40 to implementation 53, wherein a distal portion of the housing includes a recess, and further comprising: a retentive member, wherein the retentive member seats within the recess, and wherein the retentive member is configured to secure the reamer within the housing.

Implementation 55: The reaming system of implementation 54, wherein the retentive member is one or more of a spring, a clip, a ring, and a fastener.

Implementation 56: The reaming system of any one of implementation 40 to implementation 55, wherein the boss drill is configured to be inserted into the drive shaft, wherein the drive shaft includes a recess, and further comprising: a retentive member, wherein the retentive member seats within the recess, and wherein the retentive member is configured to secure the boss drill within the drive shaft.

Implementation 57: The reaming system of any one of implementation 40 to implementation 56, wherein the drive shaft is cannulated.

Implementation 58: The reaming system of implementation 57, further comprising: a guide wire, wherein the guide wire is configured to be inserted through the drive shaft.

Implementation 59: The reaming system of any one of implementation 40 to implementation 58, wherein a distal portion of the housing is at angle with respect to a proximal portion of the housing, and wherein the angle provides the rotational axis of the reamer.

Implementation 60: The reaming system of any one of implementation 40 to implementation 59, wherein the first axis of rotation is coincident with the longitudinal axis.

Implementation 61: The reaming system of any one of implementation 40 to implementation 60, wherein a maximum diameter of the reamer is greater than a maximum diameter of the boss drill.

Implementation 62: The reaming system of any one of implementation 40 to implementation 61, wherein the boss drill includes a shank and a cutting element, wherein the cutting element is located at a distal end of the shank, and wherein the shank includes a mating mechanism.

Implementation 63: The reaming system of any one of implementation 40 to implementation 62, wherein an inner surface of the drive shaft includes a ledge, and wherein a shank of the drive shaft seats on the ledge when the boss drill is coupled to the drive shaft to limit a depth into the drive shaft which the shank can extend.

Implementation 64: A reaming system comprising: a reamer assembly comprising: a housing forming a channel extending therethrough and defining a longitudinal axis, a drive shaft at least partially positioned in the channel and configured to rotate about a first axis of rotation, a reamer configured to configured to be removably coupled to the housing such that the drive shaft (i) engages the reamer and (ii) is configured to cause the reamer to rotate about a second axis of rotation that is different than the first axis of rotation, and wherein an intersection of the first axis of rotation and the second axis of rotation is located within the reamer, and a handle for use with the reamer assembly comprising: a body, a movable protrusion movably coupled to the body and configured to be moved between an extended position in which the movable protrusion extends beyond the body and a retracted position, a lever coupled to the movable protrusion and configured to move the movable protrusion between the extended position and the retracted portion, and a connection portion configured to interface with a reamer assembly.

Implementation 65: The reaming system of implementation 64, wherein the handle further comprises: a rod located within a channel of the body, wherein the movable protrusion is secured to the rod and a biasing member configured to bias the rod such that the movable protrusion is biased in the extended position.

Implementation 66: The reaming system of implementation 65, wherein the rod includes an end portion having a smaller diameter than an upper portion of the rod, wherein the biasing member seats on the end portion.

Implementation 67: The reaming system of any one of implementation 65 to implementation 66, wherein the biasing member is a spring.

Implementation 68: The reaming system of any one of implementation 65 to implementation 67, wherein the rod includes an aperture and the lever includes a lever aperture, and further comprising: a pin configured to couple the lever to the rod via the aperture and the lever aperture Implementation 69:

Implementation 70: The reaming system of any one of implementation 64 to implementation 69, wherein the body includes a least one flat surface, and wherein the lever is located on one of the at least one flat surfaces

Implementation 71: The reaming system of any one of implementation 64 to implementation 70, wherein the body includes a plurality of elongated apertures, and wherein the lever is coupled to the movable protrusion through one of the plurality of apertures.

Implementation 72: The reaming system of any one of implementation 64 to implementation 71, wherein the body and the connection portion are monolithically formed.

Implementation 73: The reaming system of any one of implementation 64 to implementation 72, wherein the connection portion is semicircular.

Implementation 74: The reaming system of any one of implementation 64 to implementation 73, wherein the connection portion includes an opening, and wherein the movable protrusion is configured to extend through the opening.

Implementation 75: The reaming system of any one of implementation 64 to implementation 74, further comprising: a boss drill configured to be removably coupled to the drive shaft such that the drive shaft is configured to cause the boss drill to rotate about the first axis of rotation.

Implementation 76: The reamer assembly of any one of implementation 64 to implementation 75, wherein a proximal end of the drive shaft is configured to be coupled to a driving tool, wherein a distal end of the drive shaft includes a ball hex, wherein the reamer includes an internal hex, and wherein the reamer is configured to mate with the drive shaft via the ball hex and the internal hex.

Implementation 77: The reamer assembly of any one of implementation 64 to implementation 76, wherein the second axis of rotation is fixed for the reamer assembly.

Implementation 78: The reamer assembly of any one of implementation 64 to implementation 77, wherein a distal portion of the housing includes a recess, and further comprising: a retentive member, wherein the retentive member seats within the recess, and wherein the retentive member is configured to secure the reamer within the housing.

Implementation 79: The reamer assembly of implementation 78, wherein the retentive member is one or more of a spring, a clip, a ring, and a fastener.

Implementation 80: The reamer assembly of any one of implementation 64 to implementation 79, wherein the boss drill is configured to be inserted into the drive shaft, wherein the drive shaft includes a recess, and further comprising: a retentive member, wherein the retentive member seats within the recess, and wherein the retentive member is configured to secure the boss drill within the drive shaft.

Implementation 81: The reamer assembly of any one of implementation 64 to implementation 80, wherein the drive shaft is cannulated.

Implementation 82: The reamer assembly of implementation 81, further comprising: a guide wire, wherein the guide wire is configured to be inserted through the drive shaft.

Implementation 83: The reamer assembly of any one of implementation 64 to implementation 82, wherein a distal portion of the housing is at angle with respect to a proximal portion of the housing, and wherein the angle provides the rotational axis of the reamer.

Implementation 84: The reamer assembly of any one of implementation 64 to implementation 83, wherein the first axis of rotation is coincident with the longitudinal axis.

Implementation 85: The reamer assembly of any one of implementation 64 to implementation 84, wherein a maximum diameter of the reamer is greater than a maximum diameter of the boss drill.

Implementation 86: The reamer assembly of any one of implementation 64 to implementation 85, wherein the boss drill includes a shank and a cutting element, wherein the cutting element is located at a distal end of the shank, and wherein the shank includes a mating mechanism.

Implementation 87: The reamer assembly of any one of implementation 64 to implementation 86, wherein an inner surface of the drive shaft includes a ledge, and wherein a shank of the drive shaft seats on the ledge when the boss drill is coupled to the drive shaft to limit a depth into the drive shaft which the shank can extend.

Implementation 88: A method of coupling a glenoid implant, the method comprising: preparing, via a reamer assembly, a glenoid of a patient to form a prepared glenoid, the reamer assembly comprising: a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, a boss drill configured to be removably coupled to the drive shaft such that the drive shaft is configured to cause the boss drill to rotate about the first axis of rotation, and coupling, to the prepared glenoid, the glenoid implant.

Implementation 89: The implementation of claim 88, wherein the glenoid implant includes a bone-facing surface featuring a convex curvature, and wherein the prepared glenoid includes a concave curvature that complements the convex curvature of the bone-facing surface of the glenoid implant.

Implementation 90: The method of implementation 88 or implementation 89, wherein the preparing the glenoid of the patient further comprises: drilling, via the boss drill of the reamer assembly, a central bore in the glenoid of the patient, wherein the glenoid implant includes a central fastener.

Implementation 91: The method of implementation 90, wherein the installing the glenoid implant further comprises: securing the central fastener of the glenoid implant to the central bore in the glenoid of the patient.

Implementation 92: The method of implementation 91, wherein the central fastener of the glenoid includes threads, and wherein the central fastener of the glenoid implant is secured to the central bore via the threads.

Implementation 93: The method of any one of implementation 90 to implementation 92, wherein the central fastener of the glenoid implant is a peg.

Implementation 94: The method of any one of implementation 64 to implementation 93, wherein the glenoid implant includes a peripheral fastener and further comprising: drilling a peripheral bore in the glenoid of the patient and securing the peripheral fastener of the glenoid implant to the peripheral bore in the glenoid of the patient.

Implementation 95: A method of coupling a glenoid implant, the method comprising: preparing, via a reamer assembly, a glenoid of a patient to form a prepared glenoid, the reamer assembly comprising: a housing forming a channel extending therethrough and defining a longitudinal axis, a drive shaft at least partially positioned in the channel and configured to rotate about a first axis of rotation, a reamer configured to configured to be removably coupled to the housing such that the drive shaft (i) engages the reamer and (ii) is configured to cause the reamer to rotate about a second axis of rotation that is different than the first axis of rotation, and wherein an intersection of the first axis of rotation and the second axis of rotation is located within the reamer, and coupling, to the prepared glenoid, the glenoid implant.

Implementation 96: The method of implementation 95, wherein the glenoid implant includes a bone-facing surface featuring a convex curvature, and wherein the prepared glenoid includes a concave curvature that complements the convex curvature of the bone-facing surface of the glenoid implant.

Implementation 97: The method of any one of implementation 95 to implementation 96, wherein the preparing the glenoid of the patient further comprises: drilling, via the boss drill of the reamer assembly, a central bore in the glenoid of the patient, wherein the glenoid implant includes a central fastener.

Implementation 98: The method of implementation 97, wherein the installing the glenoid implant further comprises: securing the central fastener of the glenoid implant to the central bore in the glenoid of the patient.

Implementation 99: The method of implementation 98, wherein the central fastener of the glenoid includes threads, and wherein the central fastener of the glenoid implant is secured to the central bore via the threads.

Implementation 100: The method of any one of implementation 97 to implementation 99, wherein the central fastener of the glenoid implant is a peg.

Implementation 101: The method of any one of implementation 95 to implementation 100, wherein the glenoid implant includes a peripheral fastener and further comprising: drilling a peripheral bore in the glenoid of the patient and securing the peripheral fastener of the glenoid implant to the peripheral bore in the glenoid of the patient.

In some implementations, a reamer assembly comprises a housing forming a channel extending therethrough and defining a longitudinal axis, a drive shaft at least partially positioned in the channel and configured to rotate about a first axis of rotation, a reamer configured to be removably coupled to the housing such that the drive shaft (i) engages the reamer and (ii) is configured to cause the reamer to rotate about a second axis of rotation that is different than the first axis of rotation, and a boss drill configured to be removably coupled to the drive shaft such that the drive shaft is configured to cause the boss drill to rotate about the first axis of rotation.

In some implementations, a reamer assembly comprises a housing forming a channel extending therethrough and defining a longitudinal axis, a drive shaft at least partially positioned in the channel and configured to rotate about a first axis of rotation, and a reamer configured to be removably coupled to the housing such that the drive shaft (i) engages the reamer and (ii) is configured to cause the reamer to rotate about a second axis of rotation that is different than the first axis of rotation, and wherein an intersection of the first axis of rotation and the second axis of rotation is located within the reamer.

In some implementations, a handle for use with a reamer assembly comprises a body, a movable protrusion movably coupled to the body and configured to be moved between an extended position in which the movable protrusion extends beyond the body and a retracted position, a lever coupled to the movable protrusion and configured to move the movable protrusion between the extended position and the retracted portion, and a connection portion configured to interface with a reamer assembly.

Those skilled in the art will recognize that a wide variety of other modifications, alterations, and combinations can also be made with respect to the above described implementations without departing from the scope of the disclosure, and that such modifications, alterations, and combinations are to be viewed as being within the ambit of the inventive concept.

1. A reamer assembly comprising:
a housing forming a channel extending therethrough and defining a longitudinal axis;
a drive shaft at least partially positioned in the channel and configured to rotate about a first axis of rotation;
a reamer configured to be removably coupled to the housing such that the drive shaft (i) engages the reamer and (ii) is configured to cause the reamer to rotate about a second axis of rotation that is different than the first axis of rotation;
a boss drill configured to be removably coupled to the drive shaft such that the drive shaft is configured to cause the boss drill to rotate about the first axis of rotation.

2. The reamer assembly of claim 1, wherein an intersection of the first axis of rotation and the second axis of rotation is located within the reamer.

3. The reamer assembly of claim 1, wherein a proximal end of the drive shaft is configured to be coupled to a driving tool, wherein a distal end of the drive shaft includes a second mating mechanism, wherein the reamer includes an internal mating mechanism, and wherein the reamer is configured to mate with the drive shaft via the second mating mechanism and the internal mating mechanism.

4. The reamer assembly of claim 1, wherein the second axis of rotation is fixed for the reamer assembly.

5. The reamer assembly of claim 1, wherein a distal portion of the housing includes a recess, and further comprising:
a retentive member, wherein the retentive member seats within the recess, and wherein the retentive member is configured to secure the reamer within the housing.

6. The reamer assembly of claim 5, wherein the retentive member is one or more of a spring, a clip, a ring, and a fastener.

7. The reamer assembly of claim 1, wherein the boss drill is configured to be inserted into the drive shaft, wherein the drive shaft includes a recess, and further comprising:
a retentive member, wherein the retentive member seats within the recess, and wherein the retentive member is configured to secure the boss drill within the drive shaft.

8. (canceled)

9. (canceled)

10. The reamer assembly of claim 1, wherein a distal portion of the housing is at angle with respect to a proximal portion of the housing, and wherein the angle provides the rotational axis of the reamer.

11. The reamer assembly of claim 1, wherein the first axis of rotation is coincident with the longitudinal axis.

12. (canceled)

13. The reamer assembly of claim 1, wherein the boss drill includes a shank and a cutting element, wherein the cutting element is located at a distal end of the shank, and wherein the shank includes a mating mechanism.

14. (canceled)

15. A reamer assembly comprising:
a housing forming a channel extending therethrough and defining a longitudinal axis;
a drive shaft at least partially positioned in the channel and configured to rotate about a first axis of rotation;
a reamer configured to be removably coupled to the housing such that the drive shaft (i) engages the reamer and (ii) is configured to cause the reamer to rotate about a second axis of rotation that is different than the first axis of rotation, and wherein an intersection of the first axis of rotation and the second axis of rotation is located within the reamer.

16. The reamer assembly of claim 15, further comprising:
a boss drill configured to be removably coupled to the drive shaft such that the drive shaft is configured to cause the boss drill to rotate about the first axis of rotation.

17. The reamer assembly of claim 15, wherein a proximal end of the drive shaft is configured to be coupled to a driving tool, wherein a distal end of the drive shaft includes a second mating mechanism, wherein the reamer includes an internal mating mechanism, and wherein the reamer is configured to mate with the drive shaft via the second mating mechanism and the internal mating mechanism.

18. The reamer assembly of claim 15, wherein the second axis of rotation is fixed for the reamer assembly.

19. The reamer assembly of claim 15, wherein a distal portion of the housing includes a recess, and further comprising:
a retentive member, wherein the retentive member seats within the recess, and wherein the retentive member is configured to secure the reamer within the housing.

20. The reamer assembly of claim 19, wherein the retentive member is one or more of a spring, a clip, a ring, and a fastener.

21. The reamer assembly of claim 15, wherein the boss drill is configured to be inserted into the drive shaft, wherein the drive shaft includes a recess, and further comprising:
a retentive member, wherein the retentive member seats within the recess, and wherein the retentive member is configured to secure the boss drill within the drive shaft.

22. (canceled)

23. (canceled)

24. The reamer assembly of claim 15, wherein a distal portion of the housing is at angle with respect to a proximal portion of the housing, and wherein the angle provides the rotational axis of the reamer.

25. The reamer assembly of claim 15, wherein the first axis of rotation is coincident with the longitudinal axis.

26. (canceled)

27. The reamer assembly of claim 15, wherein the boss drill includes a shank and a cutting element, wherein the cutting element is located at a distal end of the shank, and wherein the shank includes a mating mechanism.

28. (canceled)

29.-101. (canceled)
